(19) United States
(12) Patent Application Publication
(10) Pub. No.: US 2012/0165658 A1
(43) Pub. Date: Jun. 28, 2012

(54) SYSTEM, ARTICLE OF MANUFACTURE, AND METHOD FOR CHARACTERIZING A MEDICAL DEVICE AND/OR ONE OR MORE SENSORS MOUNTED THEREON

(76) Inventors: Jiazheng Shi, SCOTTSDALE, AZ (US); Valtino X. Afonso, OAKDALE, MN (US); Dennis J. Morgan, CRYSTAL, MN (US); Jeffrey A. Schweitzer, ST. PAUL, MN (US)

(21) Appl. No.: 12/979,663

(22) Filed: Dec. 28, 2010

(51) Int. Cl. A61B 5/05

(52) U.S. Cl. 600/424

(57) ABSTRACT

Systems and methods for characterizing a medical device and/or the sensors thereof are provided. A system comprises an electronic control unit (ECU) configured to acquire first and second configurations of the device. The ECU is configured to process the configurations to calculate an index used to characterize the device and/or the sensors thereof. An article of manufacture comprises a computer-readable storage medium having a computer program encoded thereon for characterizing the device and/or the sensor thereof. The program includes code for acquiring first and second configurations of the device, and processing them together to calculate an index used to characterize the device and/or the sensors thereof. A method for characterizing the device and/or the sensors thereof comprises providing an ECU, acquiring, by the ECU, first and second configurations of the device, and processing them together to calculate an index used to characterize the device and/or the sensors thereof.

### BACKGROUND OF THE INVENTION

a. Field of the Invention

This disclosure relates to the characterization of a medical device and/or one or more sensors mounted thereon. More particularly, this disclosure relates to a system, article of manufacture, and method for characterizing a medical device and/or one or more sensors mounted thereon.

b. Background Art

It is known that in certain conventional visualization, navigation, and/or mapping systems used, for example, in the visualization, navigation, and/or mapping of anatomical structures and/or medical devices, users of the system (e.g., clinicians) must define information relating to the configuration of a medical device used in conjunction with the system (user-defined configuration). The user-defined configuration comprises information relating to various attributes of the medical device. For example, the attributes may include the lengths of positioning sensors mounted on the device, the distance of the end-to-end spacing between adjacent positioning sensors, the distance of the midpoint-to-midpoint spacing between positioning sensors, the diameter of the medical device, and the like. With respect to these attributes, the information provided by the user, and therefore, defined by the user, comprises the magnitudes of the attributes. The user may provide the requisite information via a user input device, such as, for example and without limitation, a keyboard, a touch screen, a keypad, a mouse, and the like, or a graphical user interface (GUI) comprising one or more user-inputable or user-selectable input fields relating to the user-defined configuration of the medical device.

One challenge associated with conventional visualization, navigation, and/or mapping systems such as these is that the user-defined configurations are prone to user error, and erroneous or incorrect configurations may adversely impact the accurate operation of the system. For example, an incorrect user-defined configuration may adversely affect, among other things, field scaling performed by the system.

Another challenge with these types of conventional visualization, navigation, and/or mapping systems is that even if the user-defined configuration is accurate, other problems or events can impact the accurate operation of the system. These problems or events may include, for example, medical devices being connected to the wrong inputs of the system, broken wires between positioning sensors and the system, medical devices being disconnected from the system, positioning sensors of the medical device being disposed within a sheath, just to name a few. If any one of these problems or events occurs during, for example, the generation of a geometric model of an anatomical structure, poor geometry models can result due to displaced points and/or due to inaccurate field scaling information.

Accordingly, the inventors herein have recognized a need for a system, article of manufacture, and method for characterizing a medical device and/or one or more sensors mounted thereon that will minimize and/or eliminate one or more of the deficiencies in conventional systems.

### BRIEF SUMMARY OF THE INVENTION

The present invention is directed to a system, article of manufacture, and method for characterizing a medical device and/or one or more sensors mounted thereon. In accordance with one aspect of the invention, an apparatus is provided comprising an electronic control unit (ECU). The ECU is configured to acquire a first configuration of a medical device having a plurality of sensors mounted thereon. The ECU is further configured to acquire a second configuration of the medical device, wherein the second configuration is a calculated configuration. The ECU is still further configured to process the first and second configurations together to calculate an index upon which a characterization of the medical device and/or one or more of the sensors thereof may be based.

In accordance with another aspect of the invention, an article of manufacture is provided. The article of manufacture comprises a computer-readable storage medium having a computer program encoded thereon for characterizing a medical device and/or one or more of a plurality of sensors mounted thereon. The computer program includes code for acquiring a first configuration of the medical device and acquiring a second configuration of the medical device, wherein the second configuration comprises a calculated configuration. The computer program further includes code for processing the first and second configurations together to calculate an index upon which a characterization of the medical device and/or one or more of the sensors mounted thereon may be based.

In accordance with yet another aspect of the invention, a method for characterizing a medical device and/or one or more sensors mounted thereon is provided. The method comprises the step of providing an electronic control unit (ECU). The method further comprises the step of acquiring, by the ECU, a first configuration of a medical device. The method still further comprises the step of acquiring, by the ECU, a second configuration of the medical device, wherein the second configuration comprises a calculated configuration. The method yet still further comprises the step of processing, by the ECU, the first and second configurations together to calculate an index upon which a characterization of the medical device and/or one or more of the sensors mounted thereon may be based.

The foregoing and other aspects, features, details, utilities, and advantages of the present invention will be apparent from reading the following description and claims, and from reviewing the accompanying drawings.

### BRIEF DESCRIPTION OF THE DRAWINGS

FIG. 1 is a diagrammatic view of a system for performing at least one of a diagnostic and a therapeutic medical procedure in accordance with present teachings.

FIG. 2 is a simplified diagrammatic and schematic view of the visualization, navigation, and/or mapping system of the system illustrated in FIG. 1.

FIG. 3 is a flow diagram illustrating an exemplary embodiment of a method of characterizing a medical device and/or one or more sensors mounted thereon in accordance with present teachings.

FIG. 4 is side view of the distal portion of an exemplary medical device in accordance with the present teachings.

FIG. 5 is a table showing exemplary magnitudes of attributes of the configuration of the medical device illustrated in FIG. 4.

FIG. 6. is a top view of the distal portion of another exemplary medical device in accordance with the present teachings.

FIG. 7 is a diagrammatic illustration of an exemplary characterization algorithm for a sensor of interest in accordance with the present teachings.

FIG. 8 is a diagrammatic illustration of another exemplary characterization algorithm for a sensor of interest in accordance with the present teachings.

FIG. 9 is a diagrammatic illustration of an exemplary characterization algorithm for a medical device having a plurality of sensors mounted thereon in accordance with the present teachings.

### DETAILED DESCRIPTION OF EMBODIMENTS OF THE INVENTION

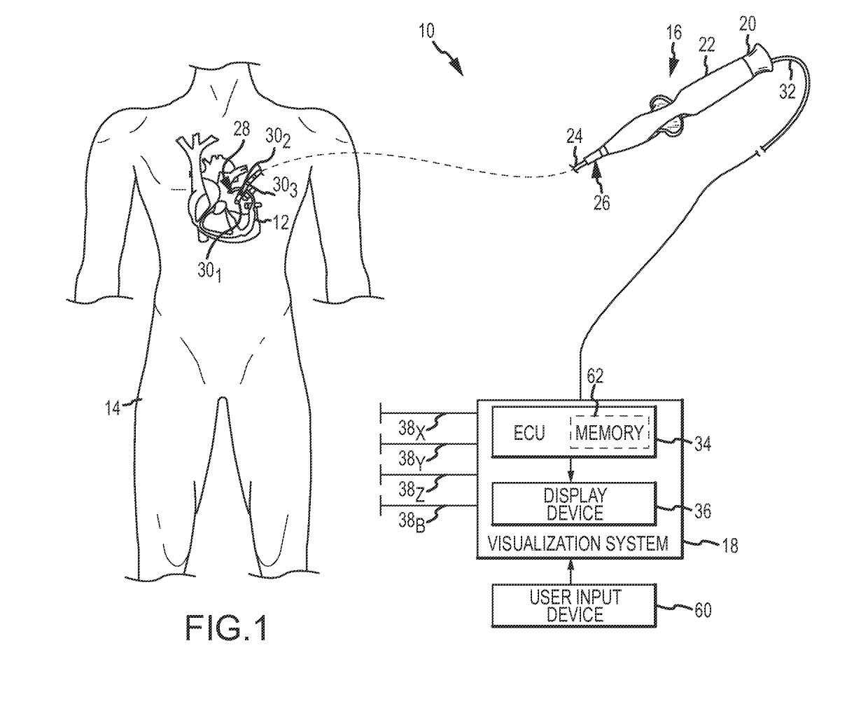

Referring now to the drawings wherein like reference numerals are used to identify identical components in the various views, FIG. 1 illustrates one exemplary embodiment of a system 10 for performing one or more diagnostic and/or therapeutic functions on or for a tissue 12 of a body 14. In an exemplary embodiment, the tissue 12 comprises heart or cardiac tissue within a human body 14. It should be understood, however, that the system 10 may find application in connection with a variety of other tissues within human and non-human bodies, and therefore, the present disclosure is not meant to be limited to the use of the system 10 in connection with only cardiac tissue and/or human bodies.

The system 10 includes a medical device 16 and a system 18 for the visualization, navigation, and/or mapping of internal body structures (hereinafter referred to as “visualization, navigation, and mapping system 18” or “system 18”). Each component of the system 10 will be described in turn below.

With continued reference to FIG. 1, in an exemplary embodiment, the medical device 16 comprises a catheter, such as, for example, an electrophysiology catheter. In other exemplary embodiments, the medical device 16 may take a form other than a catheter, such as, for example and without limitation, a sheath or catheter-introducer, or a catheter other than an electrophysiology catheter. For clarity and illustrative purposes only, the description below will be limited to an embodiment of the system 10 wherein the medical device 16 comprises a catheter (catheter 16). It will be appreciated, however, that in other embodiments, the system 10 may comprise medical devices other than a catheter, and therefore, the present disclosure is not meant to be limited to an embodiment wherein the medical device 16 of the system 10 comprises a catheter.

The catheter 16 is provided for examination, diagnosis, and/or treatment of internal body tissues such as the tissue 12. The catheter 16 may include a cable connector or interface 20, a handle 22, a shaft 24 having a proximal end 26 and a distal end 28 (as used herein, “proximal” refers to a direction toward the end of the catheter 16 near the handle 22, and “distal” refers to a direction away from the handle 22), and one or more sensors and/or electrodes mounted in or on the shaft 24 of the catheter 16. In an exemplary embodiment, one or more of the sensors or electrodes of the catheter 16 are disposed at or near the distal end 28 of the shaft 24, and comprise positioning sensors 30 (e.g., positioning electrodes or magnetic sensors (e.g., coils)) used, for example and as will be described in greater detail below, with the visualization, navigation, and mapping system 18. The catheter 16 may further include other conventional components such as, for example and without limitation, temperature sensors, additional electrodes and corresponding conductors or leads, and/or ablation elements (e.g., ablation electrodes, high intensity focused ultrasound ablation elements, and the like).

The connector 20 provides mechanical and electrical connection(s) for one or more cables 32 extending from, for example, the visualization, navigation, and mapping system 18 to the sensor(s) 30. In other exemplary embodiments, the connector 20 may also provide mechanical, electrical, and/or fluid connections for cables extending from other components in the system 10, such as, for example, an ablation system and a fluid source (when the catheter 16 comprises an irrigated catheter). The connector 20 is conventional in the art and is disposed at the proximal end 26 of the catheter 16.

The handle 22 provides a location for a user to hold the catheter 16 and may further provide means for steering or guiding the shaft 24 within the body 14. For example, the handle 22 may include means to manipulate one or more steering wires extending through the catheter 16 to the distal end 28 of the shaft 24 to steer the shaft 24. The handle 22 is also conventional in the art and it will be understood that the construction of the handle 22 may vary. In another exemplary embodiment, the control of the catheter 16 may automated (e.g., the catheter may be robotically driven or controlled, or driven and controlled by a magnetic-based guidance system). Accordingly, rather than a user manipulating a handle to steer or guide the catheter 16, and the shaft 24 thereof, in particular, an automated system is used to manipulate the catheter 16. Catheters controlled either manually or automatically are both within the spirit and scope of the present disclosure.

The shaft 24 is an elongate, tubular, flexible member configured for movement within the body 14. The shaft 24 supports, for example and without limitation, the sensors 30, associated conductors, and possibly additional electronics used for signal processing or conditioning. The shaft 24 may also permit transport, delivery and/or removal of fluids (including irrigation fluids, cryogenic ablation fluids, and bodily fluids), medicines, and/or surgical tools or instruments. The shaft 24 may be made from conventional materials such as polyurethane, and defines one or more lumens configured to house and/or transport electrical conductors, fluids, or surgical tools. The shaft 24 may be introduced into a blood vessel or other structure within the body 14 through a conventional introducer. The shaft 24 may then be steered or guided through the body 14 to a desired location such as the tissue 12 using means well known in the art.

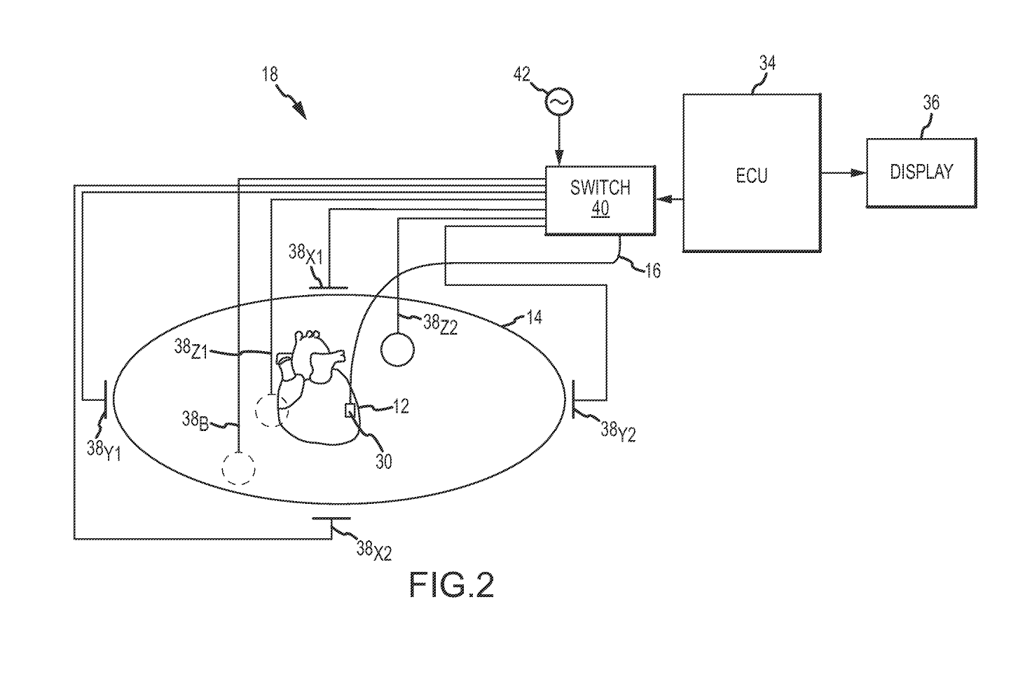

With reference to FIGS. 1 and 2, the visualization, navigation, and mapping system 18 will be described. The system 18 is provided for visualization, navigation, and/or mapping of internal body structures and/or medical devices. In an exemplary embodiment, the system 18 comprises an electronic control unit (ECU) 34 and a display device 36. Alternatively, one or both of the ECU 34 and display device 36 may be separate and distinct from, but electrically connected to and configured for communication with, the system 18.

The visualization, navigation, and mapping system 18 may comprise an electric field-based system, such as, for example, that having the model name EnSite NavX™ and commercially available from St. Jude Medical., Inc. and as generally shown with reference to U.S. Pat. No. 7,263,397 titled “Method and Apparatus for Catheter Navigation and Location and Mapping in the Heart,” the entire disclosure of which is incorporated herein by reference, or the EnSite Velocity™ system running a version of the NavX™ software. In other exemplary embodiments, however, the system 18 may comprise systems other than electric field-based systems. For example, the system 18 may comprise a magnetic field-based system such as the Carto™ system commercially available from Biosense Webster, and as generally shown with reference to one or more of U.S. Pat. No. 6,498,944 entitled “Intrabody Measurement;” U.S. Pat. No. 6,788,967 entitled “Medical Diagnosis, Treatment and Imaging Systems;” and U.S. Pat. No. 6,690,963 entitled “System and Method for Determining the Location and Orientation of an Invasive Medical Instrument,” the disclosures of which are incorporated herein by reference in their entireties. In another exemplary embodiment, the system 18 may comprise a magnetic field-based system such as the gMPS system commercially available from MediGuide Ltd., and as generally shown with reference to one or more of U.S. Pat. No. 6,233,476 entitled “Medical Positioning System;” U.S. Pat. No. 7,197,354 entitled “System for Determining the Position and Orientation of a Catheter;” and U.S. Pat. No. 7,386,339 entitled “Medical Imaging and Navigation System,” the disclosures of which are incorporated herein by reference in their entireties. In yet another embodiment, the system 18 may comprise a combination electric field-based and magnetic field-based system, such as, for example and without limitation, the Carto 3™ system also commercially available from Biosense Webster, and as generally shown with reference to U.S. Pat. No. 7,536,218 entitled “Hybrid Magnetic-Based and Impedance Based Position Sensing,” the disclosure of which is incorporated herein by reference in its entirety. In yet still other exemplary embodiments, the system 18 may comprise or be used in conjunction with other commonly available systems, such as, for example and without limitation, fluoroscopic, computed tomography (CT), and magnetic resonance imaging (MRI)-based systems.

In an exemplary embodiment, and as briefly described above, the catheter 16 includes a plurality of positioning sensors 30 for producing signals indicative of catheter position and/or orientation information. As set forth above, the positioning sensor 30 may include positioning electrodes, in the case of electric field-based systems, or alternatively, magnetic sensors (e.g., coils) configured to detect one or more characteristics of a low-strength magnetic field, for example, in the case of magnetic field-based systems. For purposes of clarity and illustration only, the system 18 will be described hereinafter as comprising an electric field-based system, such as, for example, the EnSite NavX™ or Velocity™ systems identified above.

With continued reference to FIGS. 1 and 2, in addition to the ECU 34 and the display device 36, the system 18 may further include a plurality of patch electrodes 38, among other components. With the exception of the patch electrode 38_{B} called a “belly patch,” the patch electrodes 38 are provided to generate electrical signals used, for example, in determining the position and orientation of the catheter 16 (e.g., positioning coordinates of positioning sensors 30 mounted on the catheter 16), and in the guidance thereof. In one embodiment, the patch electrodes 38 are placed orthogonally on the surface of the body 14 and are used to create axes-specific electric fields within the body 14. For instance, in one exemplary embodiment, patch electrodes 38_{X1}, 38_{X2} may be placed along a first (x) axis. Patch electrodes 38_{Y1}, 38_{Y2} may be placed along a second (y) axis, and patch electrodes 38_{Z1}, 38_{Z2} may be placed along a third (z) axis. Each of the patch electrodes 38 may be coupled to a multiplex switch 40. In an exemplary embodiment, the ECU 34 is configured, through appropriate software, to provide control signals to switch 40 to thereby sequentially couple pairs of electrodes 38 to a signal generator 42. Excitation of each pair of electrodes 38 generates an electrical field within body 14 and within an area of interest such as tissue 12. Voltage levels at non-excited electrodes 38, which are referenced to the belly patch 38_{B}, are filtered and converted and provided to ECU 34 for use as reference values.

As briefly discussed above, the catheter 16 includes one or more positioning sensors 30 configured to be electrically coupled to the ECU 34. With the positioning sensors 30 electrically coupled to the ECU 34, the positioning sensors 30 are placed within electrical fields created in the body 14 (e.g., within the heart) by exciting the patch electrodes 38. The positioning sensors 30 experience voltages that are dependent on the respective locations between the patch electrodes 38 and the respective positions of the positioning sensors 30 relative to the tissue 12. Voltage measurement comparisons made between the positioning sensors 30 and the patch electrodes 38 can be used to determine the position of each positioning sensor 30 relative to the tissue 12. Accordingly, the ECU 34 is configured to determine position coordinates (x, y, z) of each positioning sensor 30. Movement of the positioning sensors 30 proximate the tissue 12 (e.g., within a heart chamber) produces information regarding the geometry of the tissue 12. This information may be used, for example, to generate models and maps of anatomical structures that may be displayed on a display device. Information received from the positioning sensors 30 can also be used to display on a display device the location and orientation of the positioning sensors 30 and/or the tip of the catheter 16 relative to the tissue 12. Accordingly, among other things, the ECU 34 of the system 18 may provide a means for generating display signals used to the control the display device 36 and the creation of a graphical user interface (GUI) on the display device 36. In addition to the above, the ECU 34 may further provide a means for controlling various components of system 10 including, but not limited to, the switch 40.

It should be noted that while in an exemplary embodiment the ECU 34 is configured to perform some or all of the functionality described above and below, in another exemplary embodiment, the ECU 34 may be separate and distinct from the system 18, and the system 18 may have another processor (e.g., another ECU) configured to perform some or all of the functionality described herein. In such an embodiment, the processor of the system 18 would be electrically coupled to, and configured for communication with, the ECU 34. However, for purposes of clarity and illustration only, the description below will be limited to an embodiment wherein ECU 34 is part of system 18 and configured to perform the functionality described herein.

The ECU 34 may comprise a programmable microprocessor or microcontroller, or may comprise an application specific integrated circuit (ASIC). The ECU 34 may include a central processing unit (CPU) and an input/output (I/O) interface through which the ECU 34 may receive a plurality of input signals including, for example, signals generated by patch electrodes 38 and the positioning sensors 30, and generate a plurality of output signals including, for example, those used to control the display device 36 and the switch 40. The ECU 34 may be configured to perform various functions, such as those described in greater detail above and below, with appropriate programming instructions or code (i.e., software). Accordingly, the ECU 34 is programmed with one or more computer programs encoded on a computer-readable storage medium for performing the functionality described herein.

In operation, the ECU 34 generates signals to control the switch 40 to thereby selectively energize the patch electrodes 38. The ECU 34 receives position signals (location information) from the catheter 16 (and particularly the positioning sensors 30) reflecting changes in voltage levels on the positioning sensors 30 and from the non-energized patch electrodes 38. The ECU 34 uses the raw positioning data produced by the patch electrodes 38 and positioning sensors 30 and corrects the data to account for respiration, cardiac activity, and other artifacts using known or hereinafter developed techniques. The corrected data, which comprises position coordinates corresponding to each of the positioning sensors 30 (e.g., (x, y, z)) may then be used by the ECU 34 in a number of ways, such as, for example and without limitation, to create a model of an anatomical structure, to map electrophysiological data on an image or model of the tissue 12 generated or acquired by the ECU 34, or to create a representation of the catheter 16 that may be superimposed on a map, model, or image of the tissue 12 generated or acquired by the ECU 34.

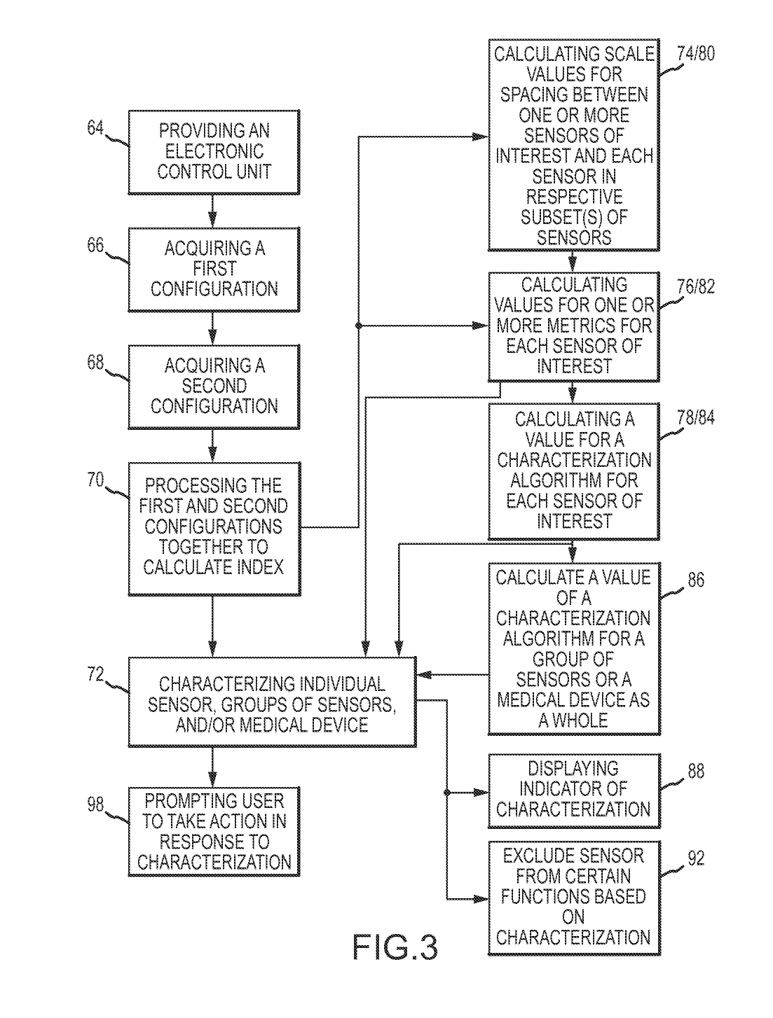

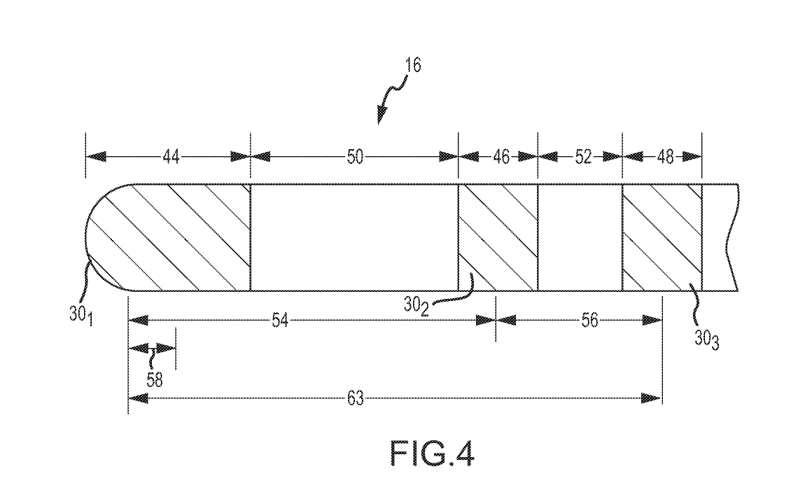

With reference to FIGS. 3 and 4, in addition to the functionality described above, in an exemplary embodiment, the ECU 34 is further configured to evaluate user-defined information relating to the configuration of the catheter 16 (“user-defined configuration”). In an exemplary embodiment, the ECU 34 is configured to continuously evaluate the user-defined configuration. Continuous, real-time evaluation may be desirable for various reasons, such as, for example, because a user of the system 10 may replace the catheter 16 with another catheter or medical device. In such an instance, the user-defined configuration provided by the user may be rendered inaccurate or incorrect, which may result in issues such as those described in the Background of the Invention section above.

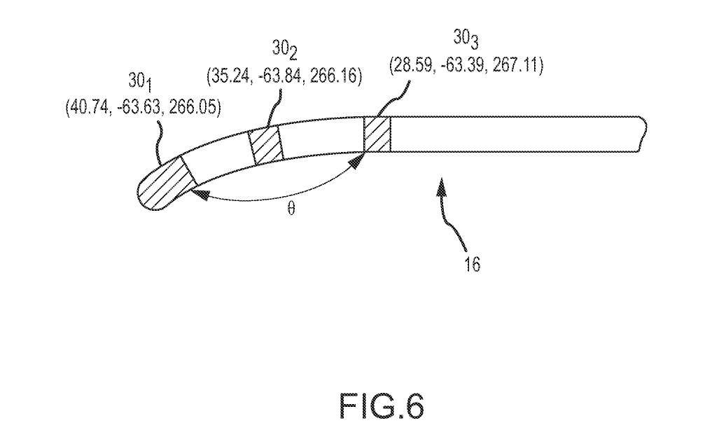

The user-defined configuration comprises information relating to attributes of the catheter 16. For example, and with reference to the exemplary catheter 16 illustrated in FIG. 4, the attributes may include the lengths of the positioning sensors 30 (e.g., the sensor 30₁ has a length 44, the sensor 30₂ has a length 46, and the sensor 30₃ has a length 48), the distance of the end-to-end spacing between adjacent positioning sensors 30 (e.g., the end-to-end spacing of the sensors 30₁ and 30₂ has a distance 50, and the end-to-end spacing of the sensors 30₂ and 30₃ has a distance 52), the distance of the midpoint-to-midpoint spacing between sensors (e.g., the midpoint-to-midpoint spacing between the sensors 30₁ and 30₂ has a distance 54, and the midpoint-to-midpoint spacing between the sensors 30₂ and 30₃ has a distance 56), the diameter and/or radius of the catheter shaft 24, the size of the catheter 16 (i.e., the French of the catheter 16), and the like. The attributes may further include the angle of the possible mechanical bend of the catheter shaft 24 at each positioning sensor 30 (See, for example, FIG. 6). With respect to each of the above identified attributes, the information relating thereto comprises magnitudes of the attributes.

It should be noted that as illustrated in FIG. 4, in certain instances, the distance of the midpoint-to-midpoint spacing between a sensor disposed at the distal tip of the shaft 24, such as sensor 30₁, and another sensor 30 (e.g., 30₂), may not be the literal midpoint-to-midpoint distance. This is because in instances wherein the front face of the sensor at the distal tip is covered by a metallic material or cap (e.g., as with ablation catheters), the measurement of the impedance is affected. Accordingly, for such sensors, the centroid thereof must be adjusted by a compensation distance 58 to compensate for this fact. In an exemplary embodiment, the magnitude of the compensation distance 58 (Comp. Dist.) is, in an exemplary embodiment, calculated by the ECU 34 using equation (1):

Comp.Dist.=0.25*r (1)

wherein “r” is the radius of the catheter shaft 24.

The information corresponding to the user-defined configuration may be as provided directly by the user, or may be derived from information provided by the user. For example, the user may define a magnitude of the midpoint-to-midpoint spacing between two adjacent positioning sensors 30, or the ECU 34 may be configured to determine the magnitude based on user-defined lengths of the two sensors, the user-defined distance of the end-to-end spacing between the two sensors 30, and/or compensation factors, such as, for example, the compensation distance 58 described above. Accordingly, the user-defined configuration may be directly provided by a user, or may be derived from other information provided by the user.

With particular reference to FIG. 3, in an exemplary embodiment, the ECU 34 is configured to acquire the user-defined configuration (Step 66). The ECU 34 may acquire the user-defined configuration in a number of ways. For example, the ECU 34 may prompt a user to provide the information at, for example, the start-up or initialization of the system 10 or the visualization, navigation, and mapping system 18. In such an embodiment, the ECU 34 is configured to receive the information from a user input device 60 associated with the ECU 34, such as, for example, the user input device 60 illustrated in FIG. 1. The user input device 60 may comprise, for example, a keyboard, a touch screen, a keypad, a mouse, a button associated with the catheter handle 22, or other like devices. In one exemplary embodiment, the user input device 60 comprises a graphical user interface (GUI) generated and displayed on a display, such as, for example, the display device 36, by the ECU 34. In such an embodiment, the ECU 34 is configured to generate a user input screen comprising one or more user-inputable or user-selectable input fields relating to the user-defined configuration of the catheter 16.

In another exemplary embodiment, the ECU 34 may acquire the user-defined configuration by retrieving previously provided information from a memory device or storage medium 62 that is part of or accessible by the ECU 34, such as, for example, the memory 62 illustrated in FIG. 1. The information comprising the user-defined configuration may have been previously provided by the user in the same manner described above using the user input device 60 and then stored in the memory 62, or the memory 62 may be pre-programmed with the information. Additionally, the user-defined configuration for the catheter 16 may be stored in a table in the memory 62 that contains user-defined configurations for a plurality of different medical devices, and the ECU 34 may be configured to acquire the correct information corresponding to a particular desired medical device from the table.

In yet another exemplary embodiment, the catheter 16 may itself include a memory such as an EEPROM that stores a user-defined configuration corresponding to that particular catheter, or stores a memory address for accessing the user-defined configuration in another memory location. The ECU 34 may acquire the user-defined configuration by retrieving the information from the appropriate memory location. Accordingly, the ECU 34 may acquire the user-defined configuration in a number of ways and/or from a number of sources, all of which are within the spirit and scope of the present disclosure.

With continued reference to FIG. 3, the ECU 34 is further configured to acquire a calculated configuration for the catheter 16 (Step 68), and, in an exemplary embodiment, to store the acquired calculated configuration in a memory or other storage medium that is part of or accessible by the ECU 34, such as, for example, the memory 62. As will be described in greater detail below, the calculated configuration may be based on position coordinates corresponding to the respective locations of each of the positioning sensors 30, or alternatively, may be calculated using various other techniques known in the art, such for example, using ultrasound.

As with the user-defined configuration described above, the calculated configuration comprises information relating to attributes of the catheter 16, such as, for example and without limitation, the distances between the positioning sensors 30. For example, and with reference to FIG. 4, the attributes may include the distance of the midpoint-to-midpoint spacing between sensors (e.g., the midpoint-to-midpoint spacing between the sensors 30₁ and 30₂ (corresponding to the distance 54 in FIG. 4), the midpoint-to-midpoint spacing between the sensors 30₂ and 30₃ (corresponding to the distance 56 in FIG. 4), and the midpoint-to-midpoint spacing between the sensors 30₁ and 30₃ (corresponding to a distance 63 in FIG. 4)). The attributes may further include the angle (θ) of the mechanical bend of the shaft 24 at various positioning sensors 30 (See FIG. 6).

The information comprising the calculated configuration may be based on and calculated using, for example, actual measured or determined position coordinates (e.g., Euclidean coordinates) of the positioning sensors 30. In another exemplary embodiment, the information comprising the calculated configuration may be based on and calculated using, at least in part, data acquired from one or more modalities, such as, for exemplary purposes only, ultrasound.

For example, in an exemplary embodiment, image data corresponding to the catheter 16, or at least the distal portion thereof, may be generated by an imaging system, such as an ultrasound-based system, and then the information corresponding to some or all of the attributes of the catheter 16 (e.g., spacing between positioning sensors 30) may be calculated or determined by processing the imaging data using known techniques. In addition, or in the alternative, the image data may be used to determine position coordinates of the positioning sensors 30. Additionally, in an exemplary embodiment, the generated data may be combined with user-defined or provided information to make the necessary calculations. For example, the data may be used to calculate the distance of the end-to-end spacing of positioning sensors, but may have to be combined with the user-defined lengths of the positioning sensors to determine the distance of the midpoint-to-midpoint spacing. Accordingly, the calculated configuration may be independent of user input, or alternatively may be dependent thereon to a certain degree. While ultrasound is specifically identified above, it will be appreciated that any modality in which position coordinates of positioning sensors or information relating to attributes of a catheter (e.g., spacing between sensors, lengths of sensors, etc.) can be acquired may be used, and therefore, remain within the spirit and scope of the present disclosure.

For purposes of clarity and illustration only, the description below will be limited to an embodiment wherein the calculated configuration is based on position coordinates of the positioning sensors 30. It will be appreciated in view of the above, however, that in other exemplary embodiments, the calculated configuration may be based on or determined using other techniques, and each of these remain within the spirit and scope of the present disclosure.

In an embodiment wherein the calculated configuration is based on positioning coordinates of positioning sensors 30, the position coordinates are measured or determined, for example, in the manner described in greater detail above (e.g., using an electric field-based, or alternatively, magnetic field-based, visualization, navigation, and mapping system 18), and may also be field scaled, unscaled, filtered, or unfiltered. For example, in an exemplary embodiment, the position coordinates may be preprocessed such that position coordinates that would clearly present issues with catheter configurations and sensor coordinates would be filtered out. This may be done in a variety of ways. For instance, measurements from the patch electrodes of the visualization, navigation, and mapping system 18 may be used to define a point that is disposed within the patient's heart, for example. Position coordinates that are substantially far away from that reference point may then be flagged or excluded from further processing since the point corresponding to those position coordinates is probably not within the heart. Alternatively, a low-order polynomial or smooth spline curve may be calculated from all of the position coordinates of the positioning sensors 30, and then the position coordinates corresponding to points that substantially deviate from the curve may be flagged or exclude from further processing. In other exemplary embodiments, however, no preprocessing of the position coordinates of the positioning sensors 30 is performed.

The ECU 34 may acquire the calculated configuration in a number of ways. For example, in one exemplary embodiment, the ECU 34 itself is configured to make some or all of the calculations used to generate the calculated configuration. In such an embodiment, the ECU 34 is configured to determine the position coordinates (Euclidean coordinates) of the positioning sensors 30 or to obtain them from another source. In either instance, the ECU 34 is configured to determine or calculate, for example, the magnitude of the spacing between at least two or more of the positioning sensors 30 based on the respective position coordinates (i.e., in an exemplary embodiment, the positioning coordinates of positioning sensors 30₁ and 30₂ are used to determine the magnitude of the spacing therebetween, the positioning coordinates of positioning sensors 30₂ and 30₃ are used to determine the magnitude of the spacing therebetween, and the positioning coordinates of positioning sensors 30₁ and 30₃ are used to determine the magnitude of the spacing therebetween). In another exemplary embodiment, however, rather than the ECU 34 performing the calculations required to generate the calculated configuration, the ECU 34 obtains the calculated configuration, or some or all of the information thereof, from another component in the system 10 or the visualization, navigation, and mapping system 18, such as, for example and without limitation, another ECU or processor of the system 10 or the visualization, navigation, and mapping system 18, an ultrasound system (or another modality) that is used with, or is part of, the system 10, or a visualization, navigation, and/or mapping system other than system 18, to name a few. Accordingly, the ECU 34 may be configured to acquire the calculated configuration in a number of ways and/or from a number of sources, all of which are within the spirit and scope of the present disclosure.

Once the ECU 34 has acquired the user-defined configuration and the calculated configuration (whether based on position coordinates of the sensors 30 or otherwise), the ECU 34 is configured to process the respective configurations together (Step 70). As will be described in greater detail below, this includes calculating an index, or in an exemplary embodiment, a plurality of indices, based on the user-defined and calculated configurations. In exemplary embodiment, the ECU 34 is further configured to make one or more characterizations relating to individual positioning sensors 30, groups of positioning sensors 30, and/or the catheter 16 as a whole based on the calculated index (Step 72). Each of these will be described in turn below.

With respect to the characterization of an individual positioning sensor of interest 30, and as briefly described above, the user-defined and calculated configurations are processed together to calculate an index. In an exemplary embodiment, the index comprises the value of one of a plurality of metrics that are dependent upon, at least in part, the scale between the user-defined and calculated configurations. These metrics include, for example and without limitation, a scale accuracy metric, a local scale similarity metric, and, in certain instances, a history metric, each of which will be described in greater detail below. Alternatively, rather than the values of one or more of these metrics comprising the index, the values may be used to calculate the index in the manner such as that described in greater detail below.

In either embodiment, wherein scale-dependent metrics are calculated, the ECU 34 is configured to calculate respective scale values for the spacing between the positioning sensor of interest 30 and each positioning sensor 30 of a subset of the positioning sensors 30 of the catheter 16 (Substep 74/80). The scale values are based on user-defined and calculated magnitudes of the distances between the positioning sensor of interest 30 and each respective positioning sensor 30 of the subset. The subset may be all or fewer than all of the positioning sensors 30 of the catheter 16 other than the positioning sensor of interest 30. In an exemplary embodiment, the subset comprises all of the positioning sensors 30 within a predetermined threshold distance (Dₜₕ) of the positioning sensor of interest 30. The threshold distance may a non-adjustable value programmed into the ECU 34 during a manufacturing process, or by the user during the start-up or initialization of the system 10 or visualization, navigation, and mapping system 18. Alternatively, the threshold distance may be adjustable by the user during use.

Each scale value “S” is calculated by the ECU 34 using equation (2):

$${S}_{ij} = \frac{{C}_{ij}}{{U}_{ij}} , \qquad ( 2 )$$

wherein “i” represents the number of the positioning sensor of interest (i.e., sensor “1” (300, sensor “2” (30₂), etc.), “j” represents the number of the positioning sensor whose spacing from the positioning sensor of interest is being evaluated, “C” is the calculated magnitude of the distance of the midpoint-to-midpoint spacing between the positioning sensors “i” and “j,” and “U” is the user-defined magnitude of the distance of the midpoint-to-midpoint spacing between the positioning sensors “i” and “j.” It will be appreciated that while the description above is with respect to the midpoint-to-midpoint spacing, in another exemplary embodiment the end-to-end spacing may be used instead. In order to better illustrate the scale value calculation, a series of scale value calculations will be described for the exemplary catheter 16 illustrated in FIG. 4.

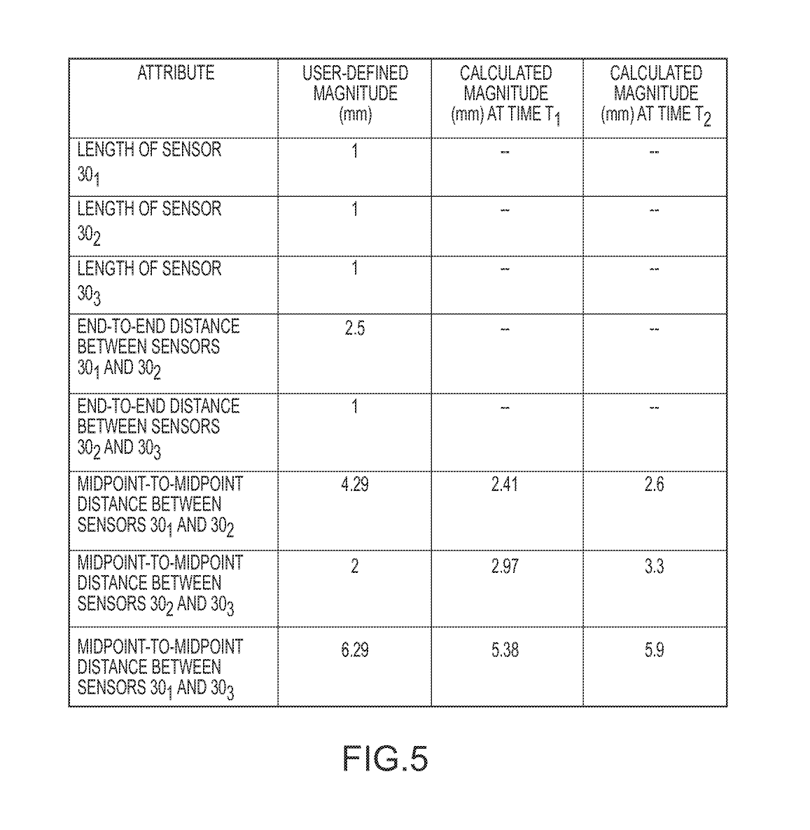

As described above, the catheter 16 illustrated in FIG. 4 comprises a plurality of positioning sensors 30 (i.e., 30₁, 30₂, and 30₃) having a number of different length and distance attributes corresponding thereto. With reference to FIGS. 4 and 5, and for purposes of illustration only, the following magnitudes have been assigned to each attribute of the user-defined configuration: length 44—2 mm; length 46—1 mm; length 48—1 mm; distance 50—2.5 mm; distance 52—1 mm; distance 54—4.29 mm; and distance 56—2 mm. For purposes of illustration only, the following magnitudes have been assigned to each attribute of the calculated configuration: distance 54—2.41 mm; distance 56—2.97 mm, and distance 63—5.38 mm. Further, for the purposes of this example, assume that positioning sensor 30₁ is the positioning sensor of interest, and therefore, respective scale values of the spacing between positioning sensors 30₁ and 30₂, and 30₁ and 30₃ will be calculated.

Accordingly, using the magnitudes of the various attributes and equation (2) above, the scale value of the spacing between the positioning sensors 30₁ and 30₂ (S₁₂) is 0.56

$$( {S}_{12} = \frac{{C}_{12}}{{U}_{12}} = \frac{2.41}{4.29} = 0.56 ) .$$

In exemplary embodiment, in order to eliminate the risk of dramatic changes in the scale value caused by even small changes in the magnitude of the user-defined distance of the spacing, a logarithm function is applied to the calculated scale (i.e., |ln(Sᵢⱼ)|). Accordingly, in the exemplary scale value calculation for S₁₂, the result of the logarithm function (i.e., |ln(0.56)|) is 0.58.

Similarly, using the various attributes and equation (2) above, the scale value of the spacing between the sensors 30₁ and 30₃ (S₁₃) is 0.85

$$( {S}_{13} = \frac{{C}_{13}}{{U}_{13}} = \frac{{C}_{13}}{{U}_{12} + {U}_{23}} = \frac{5.38}{6.29} = 0.85 ) .$$

The result of the logarithm function applied to the calculated scale value (i.e., |ln(0.85)|=0.16).

Once all the required scale values have been calculated (i.e., the scale values between the positioning sensor of interest 30 and those within the subset of positioning sensors 30 being taken into consideration), the ECU 34 is configured to calculate values for one or more metrics relating to the positioning sensor of interest 30 (Substep 76/82). As set forth above, any one of the values of the calculated metrics may comprise the index used in the characterization of the positioning sensor of interest 30 briefly described above and described in greater detail below. The metrics are dependent upon one or more of the calculated scale values (i.e., the absolute values of the natural logs of the scale values, in particular) and, in an exemplary embodiment, utilize the Sigmoid Function to transform the scale values from a varying range to a normalized range of [0,1].

One exemplary metric that may be used is “scale accuracy.” This metric measures the similarity of the user-defined and calculated distance(s) of the spacing between a sensor of interest and a set of sensors in a given predefined spatial region. In an exemplary embodiment, the scale accuracy metric is calculated by the ECU 34 using equation (3):

$${Likelihood}_{SA} = \frac{2}{1 + exp ( β \cdot {min}_{j} \{ \vert ln ( {s}_{ij} ) \vert : {U}_{ij} < {D}_{th} \} )} \qquad ( 3 )$$

In equation (3), “β” is a constant used to adjust the sharpness of the assessment. More particularly, the higher the value of β, the sharper, more strict, and less forgiving the assessment. The term

$$“ {min}_{j} \{ \vert ln ( {s}_{ij} ) \vert \} , ”$$

is the minimum of all of the calculated scale values (the absolute values of the natural logs of the calculated scale values) for positioning sensors 30 disposed within the predefined spatial region (i.e., positioning sensors 30 disposed a distance from the positioning sensor of interest 30 less than the defined threshold distance (Dₜₕ)). The distance used to assess whether a positioning sensor 30 is within the threshold distance, and therefore, the spatial region, is the user-defined distance of the spacing between that positioning sensor 30 and the positioning sensor of interest 30 (i.e., “U”). Accordingly, if the threshold distance is 10 mm and there are two positioning sensors 30 disposed less than 10 mm from the positioning sensor of interest 30, the calculated scale values of the respective distances between the positioning sensor of interest 30 and each of the two positioning sensors 30 would be compared with each other and the lowest calculated scale value (minimum) would be used in the calculation of this metric. Using equation (3), the higher the calculated value (i.e., likelihood value), the greater the similarity between the user-defined and calculated distances.

With reference to FIGS. 4 and 5, and using the user-defined and calculated magnitudes assigned to the distances of the spacing between positioning sensors 30 set forth above and in FIG. 5, an illustration of the calculation the scale accuracy metric using equation (3) will now be provided. For purposes of this illustration, the positioning sensor of interest 30 is positioning sensor 30₁, the term β is assigned a value of 3.0, and the threshold distance (Dₜₕ) is 10 mm, so as to allow for all three positioning sensors 30 of the catheter 16 illustrated in FIG. 4 to be taken into consideration.

First, respective scale values for the distances between positioning sensor 30₁ and each of the positioning sensors 30₂ and 30₃ are calculated and a logarithm function is applied to each. Accordingly, using equation (2) above with the exemplary magnitudes of the various attributes set forth in FIG. 5, the respective scale values are calculated as follows:

$${S}_{12} = \frac{2.41}{4.29} = 0.56 \to \vert ln ( 0.56 ) \vert = 0.58 ; and$$

$${S}_{13} = \frac{5.38}{4.29 + 2.0} = \frac{5.38}{6.29} = 0.85 \to \vert ln ( 0.85 ) \vert = 0.16 .$$

Next, the minimum of the absolute values of the natural logs of the calculated scale values is determined. In this example, the minimum value is 0.16. Once that value is determined, equation (3) can be performed to determine a likelihood value for the scale accuracy for the positioning sensor 30₁ as follows:

$${Likelihood}_{SA} = \frac{2}{1 + exp ( 3 \cdot 0.16 )} = \frac{2}{1 + exp ( 0.48 )} = \frac{2}{2.6} = 0.77$$

Once calculated, this value may be used in a number of ways. For example, as briefly described above, the value may comprise the index used to characterize the positioning sensor of interest 30 in the manner described in greater detail below. More particularly, the value may be used to characterize the similarity of the user-defined and calculated configurations relative to the positioning sensor of interest 30 (i.e., whether the similarity is “good,” “bad,” or in between (i.e., whether there is a high or low degree of similarity). Alternatively, and as also described in greater detail below, the value may be used to calculate the index used in the characterization.

It will be appreciated that while in an exemplary embodiment the scale accuracy metric is calculated using the particular form of equation (3) above, in other exemplary embodiments different forms of equation (3) (e.g., forms of equation (3) having operations other than

$$“ {min}_{j} \{ . \} ” ) ,$$

or equations other than equation (3) may be used, and therefore, remain within the spirit and scope of the present disclosure.

Another exemplary metric that may be used is “local scale similarity.” This metric takes into account the fact that while the field generated by the visualization, navigation, and mapping system 18 to determine the position coordinates of the positioning sensors 30 may be inhomogeneous and anisotropic on a macroscopic scale, the field may have a certain level of similarity within a local spatial region (i.e., region defined by the threshold distance Dₜₕ, for example). Accordingly, this metric measures the similarity of the user-defined and calculated distance(s) of the inter-sensor spacing between a positioning sensor of interest and a set of positioning sensors within a predefined spatial region.

In an exemplary embodiment, the local scale similarity metric is calculated using equation (4):

$${Likelihood}_{LSS} = \frac{2}{1 + exp ( β \cdot {std}_{j} \{ \vert ln ( {s}_{ij} ) \vert : {U}_{ij} < {D}_{th} \} )} . \qquad ( 4 )$$

As with equation (3), in equation (4), “β” is a constant used to adjust the sharpness of the assessment. More particularly, the higher the value of β, the sharper, more strict, and less forgiving the assessment. The term

$$“ {std}_{j} \{ \vert ln ( {s}_{ij} ) \vert \} ”$$

denotes the standard deviation of all of the calculated scale values (the absolute values of the natural logs of the calculated scale values) for positioning sensors disposed within a local spatial region relative to the positioning sensor of interest 30 defined by the threshold distance Dₜₕ (i.e., those positioning sensors 30 disposed a distance from the positioning sensor of interest 30 less than the defined threshold distance (Dₜₕ)). As with the scale accuracy metric described above, the distance used to assess whether a positioning sensor 30 is within the threshold distance, and therefore, the local spatial region, is the user-defined distance of the spacing between that positioning sensor 30 and the positioning sensor of interest 30 (i.e., “U”). Accordingly, if the threshold distance is 10 mm and there are two positioning sensors 30 disposed less than 10 mm from the positioning sensor of interest 30, the standard deviation would be calculated based on the calculated scale values (i.e., the absolute values of the natural logs of the calculated scale values) corresponding to the respective distances between the positioning sensor of interest 30 and each of the two positioning sensors 30. Using equation (4), the higher the calculated value (i.e., likelihood value), the greater the similarity between the user-defined and calculated inter-sensor distances.

With reference to FIGS. 4 and 5, and using the user-defined and calculated magnitudes assigned to the distances of the spacing between sensors set forth above and in FIG. 5, an illustration of the calculation the local scale similarity metric using equation (4) will now be provided. For purposes of this illustration, the positioning sensor of interest 30 is sensor 30₁, the term β is assigned a value of 3.0, and the threshold distance (Dₜₕ) is 10 mm, so as to allow for all three positioning sensors of the catheter 16 illustrated in FIG. 4 to be taken into consideration.

First, respective scale values for the distances between positioning sensor 30₁ and each of the positioning sensors 30₂ and 30₃ are calculated and a logarithm function is applied to each. Accordingly, using equation (2) above with the exemplary magnitudes of the various attributes set forth in FIG. 5, the respective scale values are calculated as follows:

$${S}_{12} = \frac{2.41}{4.29} = 0.56 \to \vert ln ( 0.56 ) \vert = 0.58 ; and$$

$${S}_{13} = \frac{5.38}{4.29 + 2.0} = \frac{5.38}{6.29} = 0.85 \to \vert ln ( 0.85 ) \vert = 0.16 .$$

Next, the standard deviation of the calculated scale values is determined. The standard deviation may be calculated using equation (5):

$$std = \sqrt{\frac{\sum {( s - \overline{s} )}^{2}}{N}} , \qquad ( 5 )$$

wherein “S” represents the natural log value of each calculated scale value, “ S̅” is the mean of the natural log values of the calculated scale values, and “N” is the number of calculated scale values. Using the calculated scale values set forth above, the standard deviation is approximately 0.2. Once that value is determined, equation (4) can be performed to determine a likelihood value for the local scale similarity for the positioning sensor 30₁ as follows:

$${Likelihood}_{LSS} = \frac{2}{1 + exp ( 3 \cdot 0.2 )} = \frac{2}{1 + exp ( 0.6 )} = \frac{2}{2.8} = 0.71$$

Once calculated, and as with the scale accuracy metric, this value may be used in a number of ways. For example, as briefly described above, the value may comprise the index used to characterize the positioning sensor of interest 30 in the manner described in greater detail below. More particularly, the value may be used to characterize the similarity of the user-defined and calculated configurations of the inter-sensor spacing relative to the positioning sensor of interest 30. Alternatively, and as also described in greater detail below, the value may be used to calculate the index used in the characterization.

It will be appreciated that while in an exemplary embodiment the local scale similarity metric is calculated using the particular form of equation (4) above, in other exemplary embodiments different forms of equation (4) (e.g., forms of equation (4) having operations other than

$$“ {std}_{j} \{ . \} ” ) ,$$

or equations other than equation (4) may be used, and therefore, remain within the spirit and scope of the present disclosure.

While the two metrics described above are dependent at least in part upon the scale between the user-defined and calculated configurations, in an exemplary embodiment, metrics based on the user-defined and calculated configurations, but not necessarily on dependent upon the scale therebetween, may be used to calculate the index or to generate values used in the index calculation. One such metric is “angle accuracy.” This metric measures the similarity of the user-defined and calculated magnitudes of the angles between a positioning sensor of interest and a set of positioning sensors in a predefined spatial region. In an exemplary embodiment, the angle accuracy metric is calculated using equation (6):

$${Likelihood}_{AA} = \frac{2}{1 + exp ( β \cdot \vert cos ( θ ) - cos ( {θ}_{U} ) \vert )} \qquad ( 6 )$$

In equation (6), “β” is a constant used to adjust the sharpness of the assessment. More particularly, the higher the value of β, the sharper, more strict, and less forgiving the assessment. The term “θ” is the deflection angle of the catheter 16 at the positioning sensor of interest 30 calculated, as will be described below, using the position coordinates of the positioning sensors 30 of the catheter 16. Finally, the term “θ_{U}” is a user-defined value of an angle of a mechanical bend of the shaft 24 of the catheter 16 for the positioning sensor of interest 30. The user-defined angle may be a non-adjustable value programmed into the ECU 34 during the manufacturing process, or by the user during the initialization of the system 10 or visualization, navigation, and mapping system 18. Alternatively, the threshold angle may be adjustable by the user during use.

In exemplary embodiment, the angle θ is calculated using the position coordinates (Euclidean coordinates) of some or all of the positioning sensors 30 of the catheter 16. The following is a description of one exemplary way in which the angle θ may be calculated. It will be appreciated, however, that angle θ may be calculated using other techniques, and therefore, the present disclosure is not meant to be limited to the single technique described herein.

For purposes of this example, let (x₁, y₁, z₁) be the position coordinates of the positioning sensor of interest 30, (xᵢ₋₁, yᵢ₋₁, zᵢ₋₁) and (xᵢ₊₁, yᵢ₊₁, zᵢ₊₁) be the position coordinates of the positioning sensors 30 to the left and right of the positioning sensor of interest 30, respectively. To determine the angle, the inner product (IP) of the coordinates, and the magnitude of the distances between the sensor of interest and the adjacent neighboring positioning sensors 30 must be calculated. The IP is calculated using equation (7):

IP=[(xᵢ₋₁−xᵢ)*(xᵢ₊₁−xᵢ)]+[(yᵢ₋₁−yᵢ)*(yᵢ₊₁−yᵢ)]+[(zᵢ₋₁−zᵢ)*(zᵢ₊₁−zᵢ)], (7)

the magnitude of distance of the spacing between sensor of interest and the adjacent sensor to the left (Dist₁) is calculated using equation (8):

$${Dist}_{1} = \sqrt{[ \begin{matrix}( ( {x}_{i - 1} - {x}_{i} ) * ( {x}_{i - 1} - {x}_{i} ) ) + \\ ( ( {y}_{i - 1} - {y}_{i} ) * ( {y}_{i - 1} - {y}_{i} ) ) + ( ( {z}_{i - 1} - {z}_{i} ) * ( {z}_{i - 1} - {z}_{i} ) )\end{matrix} ]} \qquad ( 8 )$$

and the magnitude of distance between positioning sensor of interest 30 and the adjacent positioning sensor 30 to the right (Dist₂) is calculated using equation (9):

$${Dist}_{2} = \sqrt{[ \begin{matrix}( ( {x}_{i + 1} - {x}_{i} ) * ( {x}_{i + 1} - {x}_{i} ) ) + \\ ( ( {y}_{i + 1} - {y}_{i} ) * ( {y}_{i + 1} - {y}_{i} ) ) + ( ( {z}_{i + 1} - {z}_{i} ) * ( {z}_{i + 1} - {z}_{i} ) )\end{matrix} ]} \qquad ( 9 )$$

Once the value for the inner product (IP) and the magnitudes of the respective distances between the positioning sensors 30 (Dist₁, Dist₂) are determined, the angle θ can be calculated using equation (10):

$$θ = arc os ( \frac{IP}{( {Dist}_{1} * {Dist}_{2} )} ) . \qquad ( 10 )$$

With reference to FIG. 6, an illustration of the calculation of the angle metric using equations (6)-(10) will now be provided for the positioning sensors 30₁-30₃ of the exemplary catheter 16 illustrated in FIG. 6. For purposes of this example, the positioning sensors 30₁, 30₂, 30₃ have been assigned the respective position coordinates reflected in FIG. 6. Further, in this example, the positioning sensor of interest is positioning sensor 30₂, the term β is assigned a value of 3.0, and the angle (θ_{U}) is assigned a value of 150°.

First, the angle θ must be calculated using, for example, equations (7)-(10) above and the position coordinates reflected in FIG. 6. More particularly, using equations (7)-(10), IP is calculated to be −36.67, Dist₁ is calculated to be 5.5, Dist₂, is calculated to be 6.7, and θ is calculated to be 174.3°. Once the angle θ is calculated, the angle metric can be calculated using equation (6) as follows:

$${Likelihood}_{AA} = \frac{2}{1 + exp ( 3.0 \cdot \vert cos ( 174.3 ) - cos ( 150 ) \vert )} = 0.81$$

Once calculated, and as with the scale accuracy and local scale similarity metrics described above, this value may be used in a number of ways. For example, as briefly described above, the value may comprise the index used to characterize the positioning sensor of interest 30 in the manner described in greater detail below. More particularly, the value may be used to characterize the similarity of the user-defined and calculated angle relative to the positioning sensor of interest 30. Alternatively, and as also described in greater detail below, the value may be used to calculate the index used in the characterization.

Yet another exemplary metric that may be used is one based on the history of one or more of the metrics described above. Accordingly, this metric may or may not be dependent upon the scale between the user-defined and calculated configurations as the scale accuracy and local scale similarity metrics are. For illustrative purposes only, the following description of this metric will be limited to an embodiment wherein the “history of the scale accuracy” metric is being considered. It will be appreciated, however, that in practice, any one of the other metrics described above may be used instead of the scale accuracy. Accordingly, history-based metrics that take into consideration metrics other than the scale accuracy metric remain within the spirit and scope of the present disclosure.

The scale of the distance between two positioning sensors should remain similar over time. Accordingly, this metric allows for the evaluation of whether the scale is remaining relatively constant, or whether there are changes that may be indicative of issues. This metric may be used periodically to compare the scale accuracy either of consecutive position samples of the positioning sensor of interest 30, or samples that are separated by a certain time interval.

In addition, or alternatively, this metric may be used a periodically to assess the change in the scale accuracy each time the catheter enters a predefined area. More particularly, a region-of-interest defined by the field generated by the visualization, navigation, and mapping system 18 may be divided into distinct volumetric spaces (e.g., the region-of-interest may be divided into a plurality of equally-sized cubes, for example) by the system 18. The system 18 is configured to monitor the position of the catheter 16 and can determine in which volumetric space the catheter 16, and one or more of the positioning sensors 30 thereof, in particular, is disposed. Using this information, each time a value for the scale accuracy metric is calculated, it may be associated with the particular volumetric space to which it corresponds. The calculated values may then be stored along with the corresponding volumetric space in, for example, a memory or storage medium associated with or accessible by the ECU 34, such as, for example, the memory 62. Thereafter, each time a value for the scale accuracy metric is calculated, the ECU 34 may retrieve or acquire a previously calculated values corresponding to the volumetric space to which the position of at least the positioning sensor of interest 30 corresponds, and for which the scale accuracy metric is currently being calculated. The two or more values corresponding to that particular volumetric space may then be used in the calculation of the history of scale accuracy metric.

In an exemplary embodiment, the history of scale accuracy metric is calculated using equation (11):

$${Likelihood}_{HS} = \frac{2}{1 + exp ( β \cdot {med}_{j} \{ \vert ln ( {s}_{ij}^{c} ) - ln ( {s}_{ij}^{p} ) \vert : {U}_{ij} < {D}_{th} \} )} \qquad ( 11 )$$

As with the other metrics described above, in equation (11), “β” is a constant used to adjust the sharpness of the assessment. More particularly, the higher the value of β, the sharper, more strict, and less forgiving the assessment. The term “ln(sᵢⱼ^{c})” is the natural log of the “current” or most recent of two or more calculated scale values for the distance between a positioning sensor of interest 30 and another positioning sensor 30, while “ln(sᵢⱼ^{p})” is the natural log of a “previous” calculated scale value for the distance between the same two positioning sensors 30. Accordingly, the term “|ln(sᵢⱼ^{c})−ln(sᵢⱼ^{p})|” is the absolute value of the change in the natural log of the calculated scale value based on the current and previous calculated scale values for the distance between the same two positioning sensors 30. The term

$${med}_{j} \{ \vert ln ( {s}_{ij}^{c} ) - ln ( {s}_{ij}^{p} ) \vert \} ”$$

is the median of all of the differences in the natural logs of the calculated scale values for positioning sensors 30 disposed a distance from the positioning sensor of interest 30 less than the defined threshold distance (Dₜₕ). The distance used to assess whether a positioning sensor 30 is within the threshold distance is the user-defined distance of the spacing between that positioning sensor 30 and the positioning sensor of interest 30 (i.e., “U”). Accordingly, if the threshold distance is 10 mm and there are two positioning sensors 30 disposed less than 10 mm from the positioning sensor of interest 30, the changes in natural logs of the calculated scale values for the respective distances between the positioning sensor of interest 30 and each of the two positioning sensors 30 would be evaluated together to determine the median value used in the calculation of this metric.

With reference to FIGS. 4 and 5, and using the user-defined and calculated magnitudes assigned to the distances of the spacing between positioning sensors 30 set forth above and reflected in FIG. 5, an illustration of the calculation the history of scale accuracy metric using equation (11) will now be provided. For purposes of this illustration, the positioning sensor of interest is sensor 30₁, the term β is assigned a value of 3.0, and the threshold distance (Dₜₕ) is 10 mm, so as to allow for all three sensors of the catheter 16 illustrated in FIG. 4 to be taken into consideration.

Respective scale values for the respective distances between the positioning sensor 30₁ and each of the positioning sensors 30₂ and 30₃ at a time t₁, must be calculated, and then a logarithm function applied to each. Accordingly, using equation (2) above with the exemplary magnitudes of the various attributes corresponding to time t₁ set forth in FIG. 5, the respective scale values are calculated as follows:

$${S}_{12} = \frac{2.41}{4.29} = 0.56 -> ln ( 0.56 ) = - 0.58 ; and$$

$${S}_{13} = \frac{5.38}{4.29 + 2.0} = \frac{5.38}{6.29} = 0.85 -> ln ( 0.85 ) = - 0.16 .$$

Respective scale values for the distances between positioning sensor 30₁ and each of the positioning sensors 30₂ and 30₃ at a time t₂ subsequent to time t₁ must also be calculated and then a logarithm function applied to each. Accordingly, using equation (2) above, with the exemplary magnitudes of the various attributes corresponding to time t₂ set forth in FIG. 5, the respective scale values are calculated as follows:

$${S}_{12} = \frac{2.6}{4.29} = 0.61 -> ln ( 0.61 ) = - 0.49 ; and$$

$${S}_{13} = \frac{5.9}{4.29 + 2.0} = \frac{5.9}{6.29} = 0.94 -> ln ( 0.94 ) = - 0.06$$

Next, absolute values of the change in the natural log of the scale value for each space between positioning sensors 30₁ and 30₂, and 30₁ and 30₃ must be calculated. Accordingly, the absolute values of the changes in the natural logs of the scale value S₁₂ and S₁₃ are 0.09 and 0.1, respectively. Once the magnitudes of the change values are determined, the median of the magnitudes of the change values may be determined. In this example, the median is 0.095. Once the median is determined, equation (11) can be performed to calculate a likelihood value for the history of scale metric for the positioning sensor 30₁ as follows:

$$\begin{matrix}{Likelihood}_{HS} = \frac{2}{1 + exp ( 3 \cdot 0.095 )} \\ = \frac{2}{1 + exp ( 0.285 )} \\ = \frac{2}{2.3} \\ = 0.87\end{matrix}$$

Once calculated, and as with the scale accuracy metric, this value may be used in a number of ways. For example, as briefly described above, the value may comprise the index used to characterize the positioning sensor of interest 30 in the manner described in greater detail below. More particularly, the value may be used to characterize the degree to which the value of the subject metric has changed relative to the positioning sensor of interest 30. Alternatively, and as also described in greater detail below, the value may be used to calculate the index used in the characterization.

It will be appreciated that while in an exemplary embodiment the history of scale accuracy metric is calculated using the particular form of equation (11) above, in other exemplary embodiments different forms of equation (11) (e.g., forms of equation (11) having operations other than

$$“ {med}_{j} \{ . \} ” ) ,$$

or equations other than equation (11) may be used, and therefore, remain within the spirit and scope of the present disclosure.

As briefly described above, once values for one or more of the aforedescribed metrics are calculated for a positioning sensor of interest, one or more of them may comprise the index used in the characterization of the positioning sensor of interest 30 described briefly above and in greater detail below. However, in another exemplary embodiment, rather than the values of the metrics comprising the index, one or more indices used for particular types of characterizations may be based on a combination of all or a subset of the calculated values of the metrics, and therefore, the values of the metrics may be used to calculate one or more indices.

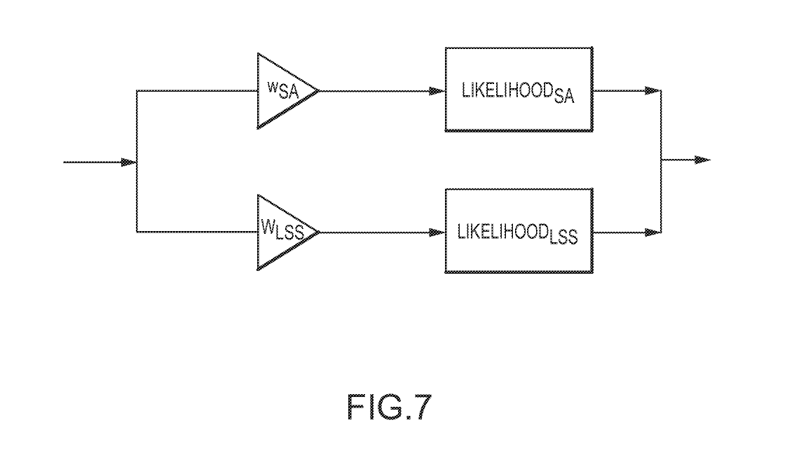

For instance, in an exemplary embodiment, the positioning sensor of interest 30 may be characterized in terms of the consistency of the user-defined configuration as compared to the calculated configuration relative to the positioning sensor of interest 30. In an exemplary embodiment, the algorithm used to calculate the index for this particular characterization may combine two or more of the metric calculations described above. For example, in the embodiment illustrated in FIG. 7, the “consistency configuration algorithm” is determined by utilizing the scale accuracy and local scale similarity metrics. More particularly, in an exemplary embodiment, the harmonic mean technique is used to combine the two metrics. As illustrated in FIG. 7, weights (i.e., w_{SA}, w_{LSS}) may be assigned to each metric in an effort to normalize the metrics being taken into consideration. The weights may be non-adjustable values preprogrammed into the ECU 34 for each metric during the manufacturing process, or by the user during the initialization of the system 10 or visualization, navigation, and mapping system 18. Alternatively, the weights may be adjustable by the user during use. In exemplary embodiment, each weight has a value between zero (0.0) and one (1.0), and the sum of the weights used in a particular equation is one (1.0). Accordingly, in view of the above, in an exemplary embodiment, the consistency characterization metric relative to the positioning sensor of interest 30 may be calculated using equation (12):

$$Likelihood {Sensor}_{Conf} = \frac{1}{\frac{{w}_{SA}}{{likelihood}_{SA}} + \frac{{w}_{LSS}}{{likelihood}_{LSS}}} \qquad ( 12 )$$

As with the metrics described above, the higher the calculated value (i.e., likelihood value), the greater the consistency between the user-defined configuration relative to the positioning sensor of interest 30, and vice versa.

It will be appreciated that while the description of the consistency characterization metric is limited to an embodiment wherein it is based solely on the scale accuracy and local scale similarity metrics, in other embodiments additional or alternative metrics, including but not limited to those described herein, may be used. Therefore, a consistency configuration metric based on metrics in addition to or other than those specifically described above remain within the spirit and scope of the present disclosure.

Using the values for the scale accuracy and local scale similarity metrics calculated above (i.e., 0.77 and 0.71, respectively) for positioning sensor 30₁, an illustration of the calculation of the consistency characterization metric using equation (12) will now be provided. For purposes of this illustration, the positioning sensor of interest 30 is once again positioning sensor 30₁ of the catheter 16 illustrated in FIG. 4, the weight assigned to the scale accuracy metric is 0.7, and the weight assigned to the local scale similarity metric is 0.3. Accordingly, in this example, the consistency configuration metric is calculated as follows:

$$Likelihood {Sensor}_{Conf} = \frac{1}{\frac{0.7}{0.77} + \frac{0.3}{0.71}} = .75$$

Once calculated, the value (i.e., likelihood value) may be used in a number of ways. For example, the value may comprise the index that the ECU 34 uses to characterize the positioning sensor of interest 30 in terms of the consistency of the user-defined configuration relative to the positioning sensor of interest 30 in the manner described in greater detail below. More particularly, the value may be used to characterize the consistency between the user-defined and calculated configurations relative to the positioning sensor of interest 30. Alternatively, and as will be described in greater detail below, the value may be used to calculate the index used to characterize a group of positioning sensors that includes the positioning sensor of interest 30, or the catheter 16 as a whole to which the positioning sensor of interest 30 is mounted.

In another exemplary embodiment, in addition to, or instead of, characterizing a positioning sensor of interest in terms of consistency, the positioning sensor of interest 30 may be characterized in terms of health or degree of confidence. For example, the health of a positioning sensor may be characterized based on the functionality of the positioning sensor (i.e., is the positioning sensor functioning properly or is it broken (i.e., a “bad” sensor)). A positioning sensor may be considered “broken” or “bad,” and therefore characterized as “unhealthy” or “bad,” for a number of reasons. For example, the positioning sensor 30 may be disconnected from an associated wire, a bend may have formed in the positioning sensor 30 thereby resulting in coagulation, and the like.

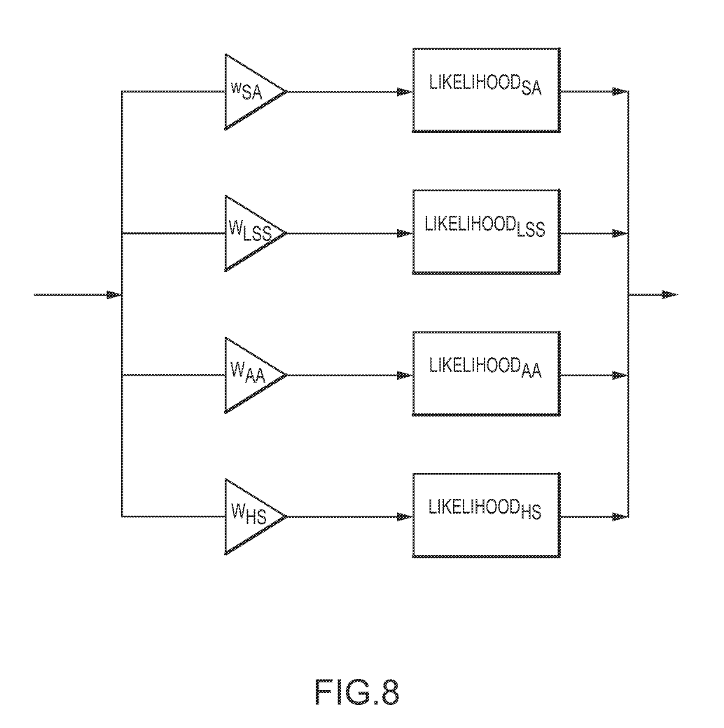

In the embodiment illustrated in FIG. 8, and provided for exemplary purposes only, all four of the above-described metrics are utilized in the algorithm used to calculate the index for characterizing the health of the positioning sensor of interest 30 in terms of functionality. More particularly, all four metrics are combined using the harmonic mean technique. As illustrated in FIG. 8, weights (e.g., w_{SA}, w_{LSS}, w_{AA}, w_{HS}) may be assigned to each metric for purposes of normalization of the metrics. The weights may be non-adjustable values preprogrammed into the ECU 34 for each metric during the manufacturing process, or by the user or system operator during the initialization of the system 10 or visualization, navigation, and mapping system 18. Alternatively, the weights may be adjustable by the user or system operator during use. In exemplary embodiment, each weight has a value between zero (0.0) and one (1.0), and the sum of the weights used in a particular equation is one (1.0).

Accordingly, in view of the functionality characterization metric relative to the positioning sensor of interest 30 may be calculated using equation (13):

$$Likelihood {Sensor}_{BAD} = \frac{1}{\begin{matrix}\frac{{w}_{SA}}{{likelihood}_{SA}} + \frac{{w}_{LSS}}{{likelihood}_{LSS}} + \\ \frac{{w}_{AA}}{{likelihood}_{AA}} + \frac{{w}_{HS}}{{likelihood}_{HS}}\end{matrix}} . \qquad ( 13 )$$

As with the metrics described above, the higher the likelihood value, the more “healthy” the corresponding positioning sensor of interest 30, and therefore, the greater the likelihood that the positioning sensor of interest 30 is functioning properly, and vice versa. This equation may be carried out in a similar manner to that described and illustrated above with respect to equation (12), and therefore, a detailed description of the calculation of this equation will not be provided. While the exemplary algorithm described above utilized all four of the above-described metrics, it will be appreciated that in other exemplary embodiments, less than all four of the above-described metrics may be used, or metrics other than those specifically identified may be used in addition to or instead of some or all of the metrics described herein. These embodiments remain within the spirit and scope of the present disclosure.

Once calculated, the value (i.e., likelihood value) may be used in a number of ways. For example, the value may comprise the index that the ECU 34 uses to characterize the positioning sensor of interest 30 in the manner described in greater detail below. More particularly, the value may be used to characterize the health or functionality of the positioning sensor of interest 30. Alternatively, and as will be described in greater detail below, the value may be used to calculate the index used to characterize a group of positioning sensors that includes the positioning sensor of interest 30, or the catheter 16 as a whole to which the positioning sensor of interest 30 is mounted.

In addition to characterizing a positioning sensor in terms of consistency or functionality, the same or different algorithm or harmonic mean formulae (i.e., taking into account the same or different subsets of metrics, weights, thresholds, and the like) may be used to characterize a positioning sensor of interest 30 in still other ways and in other terms.

For example, in another exemplary embodiment, an index may be calculated for a characterization algorithm used to characterize a positioning sensor of interest 30 in terms of location within an anatomical structure or the occurrence of an event with respect to the positioning sensor of interest 30. Exemplary events may include, for example, the positioning sensor of interest 30 being disposed within a chamber of the heart, in the pulmonary vein, or out of the body. Based on the location characterization, the health of the positioning sensor may be further characterized. For example, if the positioning sensor of interest 30 is characterized as being located outside of the body, it may be characterized as being “unhealthy,” while if it is characterized as being within a desired anatomical structure, it may be characterized as “healthy.”

In another exemplary embodiment, an index may be calculated for a characterization algorithm used to characterize the disposition of the positioning sensor of interest relative to a medical device, such as, for example, a sheath, used in conjunction with the catheter 16 (i.e., is the positioning sensor in or out of the sheath). Based on the disposition characterization, the health of the positioning sensor may be further characterized. For example, if the positioning sensor of interest 30 is characterized as being disposed with a sheath, it may be characterized as being “unhealthy,” while if it is characterized as being outside of the sheath, it may be characterized as “healthy.”

In either of these embodiments, the calculated likelihood values may be calculated and used in numerous ways, including those described above with respect to the likelihood values calculated for the consistency and health/functionality algorithms described above. Thus, it will be appreciated that using some or all of the metrics described above, many different characterization metrics or algorithms can be calculated, and characterizations based on those characterization metrics made, all of which remain within the spirit and scope of the present disclosure.

Accordingly, in view of the above, any number of indices may be calculated to characterize a positioning sensor of interest in any number of ways. In an exemplary embodiment, once an index (or in an exemplary embodiment, multiple indices) is calculated, the ECU 34 is configured to characterize the positioning sensor of interest based on the index. For instance, in an exemplary embodiment, the index relates the characterization of the positioning sensor of interest 30 in terms of the consistency of the user-defined configuration compared to the calculated configuration.

One way in which the characterization of the positioning sensor of interest 30 may be carried out is by comparing the index to one or more threshold values corresponding to one or more levels of consistency, and then, based on whether the calculated value meets, exceeds, or falls below the threshold(s), determining the level of consistency relative to the positioning sensor of interest 30. The threshold value(s) may be preset, non-adjustable value(s) programmed into the ECU 34 during the manufacturing process, or by the user during the initialization of the system 10 or visualization, navigation, and mapping system 18. The preset value(s) may be based on, for example, a previously conducted clinical studies. Alternatively, the threshold value may be adjustable by the user during use of the system 10 to vary the sensitivity of the characterization of the positioning sensor of interest 30.

In another exemplary embodiment, the ECU 34 may be configured to look-up the calculated likelihood value in a look-up table stored in a storage medium or memory that is part of accessible by the ECU 34, such as the memory 62, and a determination with respect to the health of the positioning sensor of interest 30 may then be made by the ECU 34. In either instance, the ECU 34 may characterize the positioning sensor of interest 30 as “consistent,” “inconsistent,” or somewhere in between based on the calculated likelihood value for that positioning sensor of interest 30.

In another exemplary embodiment, in addition to, or rather than, comparing the calculated index to one or more thresholds or looking up the index in a look-up table, the ECU 34 may be trained to automatically characterize the consistency of the positioning sensor of interest 30 based on the calculated index. The ECU 34 may be trained in a number of ways. For example, clinical data deemed to be “true” may be collected and used to train the ECU 34 to allow it make the correct characterizations. In another embodiment, during the actual use of the system 10, the user or another operator of the system may provide feedback to the ECU 34 to indicate the level of consistency of the positioning sensor of interest 30. Based on this input, the ECU 34 may learn that certain values or ranges of values of the index correspond to certain consistency levels of the positioning sensor of interest 30.

While the description above is limited to the characterization of the positioning sensor of interest 30 in terms of consistency, other characterizations based on other indices, such as, for example, those described above (e.g., those based on the scale accuracy, local scale similarity, angle accuracy, and history metrics described above, or those based on the “health” algorithms (e.g., functionality, location, disposition of the positioning sensor of interest 30), for example), may be made in the same or similar manner. Accordingly, the description above applies with equal force to each of the other types of characterizations and will not be repeated for each.

In addition to characterizing sensor of interest 30, the ECU 34 may be further configured to utilize the characterization of the positioning sensor of interest 30 in a number of ways. For example, in the instance wherein the characterization of the positioning sensor of interest 30 relates to the consistency, an alert or indicator may be provided to the user based on the characterization of the consistency (Step 88). The alert may take the form of a visual indicator, such as, for example, a light or a message displayed on, for example, the display 36, and/or an audio alert in the form of a buzzer, alarm, audible message, or other similar indicator may be activated. In an exemplary embodiment, the alert may only be provided if the characterization is one of an inconsistent sensor (i.e., no alert is provided if the sensor is characterized as “consistent”). Alternatively, an alert may be provided regardless of whether the characterization is one of consistent or inconsistent, with different forms of visual indicators or alerts being used for the different characterizations. In any instance, the ECU 34 is configured to determine the alert(s) to be provided, and to then generate a signal(s) representative of the corresponding alert(s). In an embodiment wherein alerts are visually displayed to the user, the ECU 34 outputs the generated signal to a display device, such as, for example, the display device 36, which then displays the alerts represented by the signal(s). In an embodiment wherein alerts additionally or alternatively comprise an audio alert, the signal is output to an audio output device (e.g., a speaker), which causes the alert(s) represented by the signal to be provided to the user.

In addition to, or instead of, the alerts described above, in an exemplary embodiment, once the index has been calculated, it may be displayed in visual form for the user or operator of the system 10 to see and interpret. In one exemplary embodiment, the index may be displayed in numerical form (e.g., a digital readout) on the display 36. This embodiment provides the user with a real-time characterization of the consistency of the positioning sensor of interest 30. Accordingly, if the ECU 34 calculates the index to be 0.71, a reading of “0.71,” for example, may be displayed on the display 36. It will be appreciated that the index value may be displayed in conjunction with an indication as to the characterization of the positioning sensor 30, or may be displayed absent the such an indication (i.e., with our without an indication as to whether the ECU 34 has characterized the positioning sensor of interest as consistent or inconsistent, for example).

In another exemplary embodiment, rather than, or in addition to, providing the visual/audio indications of the positioning sensor characterizations set forth above, the ECU 34 may be configured to use the consistency characterization in other ways. For example, in an instance wherein the positioning sensor 30 of interest is deemed to be consistent, the position coordinates of the positioning sensor 30 may be continuously monitored in real-time with an independent thread running on the ECU 34 in the background.

Alternatively, in an instance wherein the positioning sensor of interest 30 is deemed to be inconsistent, the ECU 34 may prompt or request that the user or system operator verify or make corrections to the user-defined configuration, to verify the calculated configuration, or to take some other corrective action (Step 90). The position coordinates of a positioning sensor of interest deemed to be “inconsistent” may also be excluded from certain functionality of the visualization, navigation, and mapping system 18, such as, for example and without limitation, geometry point collection for model construction, electrogram data collection for diagnostic landmark maps, map labels, lesion markers, and the like (Step 92). Similarly, the position coordinates of such inconsistent positioning sensors 30 may also be excluded from functionality, such as, for example, field scaling, among others, of the system 18. In another exemplary embodiment wherein the positioning sensor 30 is deemed to be inconsistent, rather than requiring the user to take corrective action, the ECU 34 may prompt the user to verify a graphical rendition of the positioning sensors or catheter 16 generated by the visualization, navigation, and mapping system 18. If the user verifies that the accuracy, then the current metric calculations may be used as a normalization factor when computing future metrics.

While the description above is limited to the utilization of a characterization in terms of consistency, other types of characterizations based on other indices, such as, for example, those described above (e.g., those based on the scale accuracy, local scale similarity, angle accuracy, and history metrics described above, or those based on the “health” algorithms (e.g., functionality, location, disposition of the positioning sensor of interest 30), for example), may be utilized in the same or similar manner. Accordingly, the description above applies with equal force to each of the other characterizations and will not be repeated for each.

Further, while the description above has been limited to the evaluation and characterization of a single positioning sensor of interest 30, it will be appreciated that the ECU 34 may be configured to evaluate and characterize more than one positioning sensor of interest 30 either simultaneously or successively using the methodology/techniques described above. Accordingly, the user-defined configuration with respect to two or more positioning sensors of interest may be evaluated at the same time, and each may be characterized independently. Therefore, the present disclosure is not meant to be limited to an embodiment wherein only one positioning sensor of interest may be evaluated at a time, rather embodiments wherein a plurality of positioning sensors of interest are considered simultaneously or otherwise remain within the spirit and scope of the present disclosure.

Additionally, and as was briefly described above, in an exemplary embodiment, the ECU 34 is further configured to evaluate and characterize groups of positioning sensors 30 or the catheter 16 as a whole. In either embodiment, each positioning sensor 30 in the group to be considered (which, in the case of the evaluation of the catheter 16 as a whole, includes all of the positioning sensors 30 of the catheter 16) is evaluated individually as described in great detail above, and then the individual likelihood values of the metrics or algorithms may be combined to allow for the characterization of the group of positioning sensors 30, or the catheter 16 as a whole.

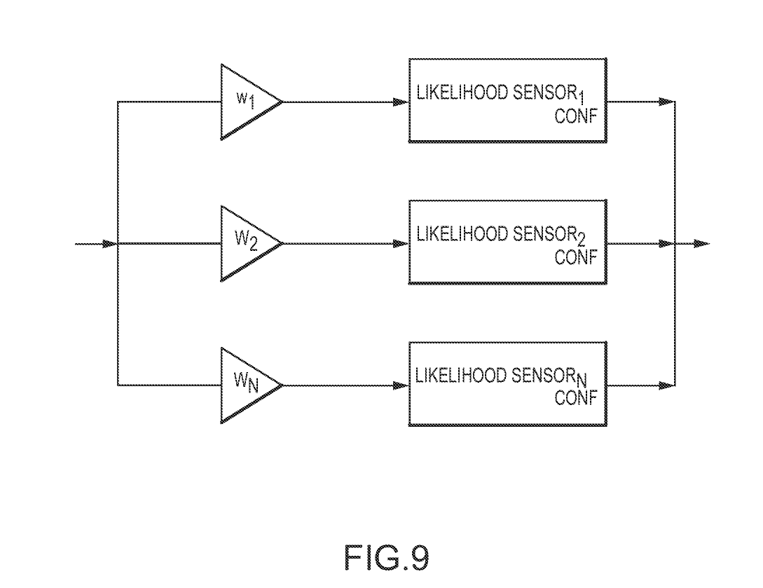

For example, likelihood values of consistency characterization algorithms may be calculated for each positioning sensor 30 in the group of positioning sensors being considered using, for example, equation (12) above. Once a likelihood value is calculated for each positioning sensor 30, the values are processed together to calculate an index, which may then be used to characterize the consistency of the group of positioning sensors 30 or the catheter 16 as a whole. With reference to FIG. 9, one way in which the individual likelihood values may be processed together is by combining them using the harmonic mean technique. The harmonic mean of the individual likelihood values, and therefore, the likelihood value of the group of positioning sensors 30 or the catheter 16 as a whole

$$( Likelihood {C}_{Conf} ) ,$$

may be calculated using equation (14):

$$Likelihood {C}_{Conf} = \frac{1}{{\sum }_{i = 1}^{n} \frac{{w}_{i}}{Likelihood {Sensor}_{Conf}}} , \qquad ( 14 )$$

wherein

$$Likelihood {Sensor}_{Conf}$$

for each positioning sensor “i” through “n” is calculated using equation (12) above, and each

$$Likelihood {Sensor}_{Conf}$$

may be assigned a weight (i.e., wᵢ) in an effort to normalize the likelihood values being taken into consideration. As with the equations described above, the weights may be non-adjustable values programmed into the ECU 34 for each likelihood value during the manufacturing process, or by the user during the initialization of the system 10 or visualization, navigation, and mapping system 18. Alternatively, the weights may be adjustable by the user during use. In exemplary embodiment, each weight has a value between zero (0.0) and one (1.0), and the sum of the weights used in a particular equation is one (1.0).

Once calculated, the likelihood value for the group of positioning sensors 30 or the catheter 16 as a whole may comprise the index, and the ECU 34 may be configured to use the index to characterize the group of positioning sensors 30 or the catheter 16 as a whole. The ECU 34 may characterize the group of positioning sensors 30 or the catheter 16 as a whole, and utilize that characterization, in the same or similar manner as that described above with respect to the consistency characterization of an individual positioning sensor. Accordingly, the description above with respect to the characterization and utilization of the same applies here with equal force and will not be repeated.

While the description above has been with respect to an embodiment wherein the catheter 16 and/or one or more positioning sensors 30 thereof is characterized based on user-defined and calculated configurations of the catheter 16, the present disclosure is not meant to be so limited. Rather, in other exemplary embodiments, the user-defined configuration may be replaced by a calculated configuration (for purposes of clarity and illustration only, referred to hereafter as “first calculated configuration”), such that the catheter 16 and/or the positioning sensors 30 thereof may be characterized based on multiple calculated configurations. For purposes of clarity and illustration only, the calculated configuration described in great detail above will hereafter be referred to as the “second calculated configuration” in order to distinguish it from the calculated configuration that replaced the user-defined configuration.

In such an embodiment, the information comprising the first calculated configuration may be determined or calculated in a manner different than that used to determine or calculate the information comprising the second calculated configuration. For example, in an embodiment wherein the information comprising the second calculated configuration is calculated using position coordinates of the positioning sensors 30 determined using electric or magnetic field-based systems, the information comprising the first calculated configuration may be determined or calculated using a different modality, such as, for example, ultrasound, as described in greater detail above. Alternatively, in an embodiment wherein the information of the second calculated configuration is calculated using position coordinates of the positioning sensors 30 measured or determined using an electric field-based system, the information of the first calculated configuration may be calculated using position coordinates of the positioning sensors 30 measured or determined using a magnetic field-based system, and vice versa.

In an embodiment wherein the information of the second calculated configuration is calculated based on position coordinates of the positioning sensors 30 measured or determined by an electric field-based system, and the information of the first calculated configuration is calculated based on position coordinates of the positioning sensors 30 measured or determined by a magnetic field-based system, the catheter 16 would further include one or more magnetic sensors (e.g., coils). The position coordinates of the magnetic sensor(s) may be determined in accordance with known techniques. Based on the position coordinates of the magnetic sensor(s) and a known arrangement of the positioning sensors 30 relative to the magnetic sensor(s) (e.g., the spacing therebetween), the position coordinates of the positioning sensors 30 may be determined. The ECU 34 may be configured to determine the position coordinates of both the magnetic sensor(s) and the positioning sensors 30 based on the position coordinates of the magnetic sensor(s), or alternatively, another component in the system may be configured to make one or both of these determinations and then communicate it to the ECU 34.

Conversely, in an embodiment wherein the information of the second calculated configuration is calculated based on position coordinates of the positioning sensors 30 measured or determined by a magnetic field-based system, and the information of the first calculated configuration is calculated based on position coordinates of the positioning sensors 30 measured or determined by a electric field-based system, the catheter 16 would further include one or more sensors (e.g., electrodes). The position coordinates of the electrode(s) may be determined in accordance with known techniques, such as, for example, those described above. Based on the position coordinates of the electrode(s) and a known arrangement of the positioning sensors 30 relative to the electrode(s) (e.g., the spacing therebetween), the position coordinates of the positioning sensors 30 may be determined. The ECU 34 may be configured to determine the position coordinates of both the electrode(s) and the positioning sensors 30 based on the position coordinates of the electrode(s), or alternatively, another component in the system may be configured to make one or both of these determinations and then communicate it to the ECU 34.

The ECU 34 may acquire the first calculated configuration in a number of ways. For example, in one embodiment, the ECU 34 is configured to make some or all of the calculations used to generate the first calculated configuration. Accordingly, the ECU 34 may be configured to generate or obtain the data (e.g., position coordinates or other required data) used to make some or all of the necessary calculations. In another exemplary embodiment, however, the ECU 34 is configured to receive or obtain the first calculated configuration, or some or all of the information thereof, from another component in the system 10 or visualization, navigation, and mapping system 18, such as, for example and without limitation, another ECU or processor of the visualization, navigation, and mapping system 18 or system 10, an ultrasound system (or another modality) that is used with, or part of, the system 10, and a visualization, navigation, and/or mapping system other than system 18, to name a few. Accordingly, the ECU 34 may acquire the first calculated configuration in a number of ways and/or from a number of sources, all of which remain within the spirit and scope of the present disclosure.

As with the embodiment described in great detail above wherein the characterization of the catheter 16 and/or one or more of the positioning sensors 30 thereof is based on user-defined and calculated configurations of the catheter 16, and irrespective of the modality used to calculate or determine the information of the first and second calculated configurations, once the ECU 34 has acquired both the first and second calculated configurations, the ECU 34 is configured to process them together. This includes calculating an index, or in an exemplary embodiment, a plurality of indices, based on the first and second calculated configurations. In an exemplary embodiment, the ECU 34 is further configured to make one or more characterizations, such as, for example, those described in great detail above, relating to individual positioning sensors 30, groups of positioning sensors 30, or the catheter 16 as a whole. In an exemplary embodiment, the first and second calculated configurations may be processed in the same manner as that described above with respect to the user-defined and calculated configurations. In such an embodiment, with exception of replacing the terms of the equations above relating to the user-defined configuration with the counterparts of the first calculated configuration, the description set forth above generally applies here with equal force and will thus not be repeated.

Accordingly, in view of the above, the characterization of the catheter 16 and/or one or more positioning sensors 30 thereof may be carried out in a number of ways, including, for example and without limitation, by taking into account user-defined and calculated configurations of the catheter 16, or multiple calculated configurations of the catheter 16, all of which remain within the spirit and scope of the present disclosure.

It will be appreciated that in addition to the structure of the system 10, and the article of manufacture described above, another aspect of the present disclosure is a method for characterizing a medical device and/or one or more of a plurality of positioning sensors mounted thereon. It will be further appreciated that the methodology and constituent steps thereof performed and carried out by the ECU 34, and described in great detail above, apply to this aspect of the disclosure with equal force. Therefore, the description of the methodology performed or carried out by the ECU 34 set forth above will not be repeated in its entirety, rather a summary will be provided.

Accordingly, with respect to FIG. 3, and in its most general form, the method includes a step 64 of providing an electronic control unit (ECU), such as, for example, the ECU 34 described above. The method further comprises a step 66 of acquiring, by the ECU, a first configuration for the medical device, which, in an exemplary embodiment, comprises a catheter, such as, for example, the catheter 16 described above. In an exemplary embodiment, the first configuration comprises a user-defined configuration, however, in other exemplary embodiments, the first configuration may comprise a calculated configuration. The method still further comprises a step 68 of acquiring, by the ECU, a second configuration for the medical device wherein the second configuration comprises a calculated configuration. In one exemplary embodiment, the second configuration is a calculated configuration based on position coordinates corresponding to the respective positions of each of the plurality of sensors, such as, for example, the positioning sensors 30 described above (e.g., positioning electrodes or magnetic sensors (e.g., coils)), mounted on the medical device. The method yet still further comprises a step 70 of processing, by the ECU, the first and second configurations together to calculate an index. The index may then be used in to characterize the medical device and/or one or more of the sensors mounted thereon. Accordingly, in an exemplary embodiment, the method further includes a step 72 of characterizing, by the ECU, the medical device and/or one or more of the sensors mounted thereon based on the calculated index.

In one exemplary embodiment, the first and second configurations each comprise magnitudes of the distances between the plurality of sensors. In this embodiment, the processing step 70 comprises a substep 74 of calculating, by the ECU, a respective scale for the spacing between a sensor of interest and each sensor of a subset of the sensors mounted on the medical device based on the respective magnitudes of the first and second configurations for the distances between the sensor of interest and each sensor of the subset of sensors. In an exemplary embodiment, the processing step 70 further comprises a substep 76 of calculating, by the ECU, a value of a metric based on one or more of the calculated scales. In one exemplary embodiment, the value of the metric comprises the index used in the characterization step 72 for characterizing the sensor of interest mounted on the medical device.

In another exemplary embodiment, the calculating substep 76 of the processing step 70 comprises calculating, by the ECU, values for a plurality of metrics, at least one of which is based on one or more of the calculated scales calculated in substep 74. In such an embodiment, the processing step 70 may still further comprise a substep 78 of calculating, by the ECU, a value for a characterization algorithm for the positioning sensor of interest based on the values of the plurality of metrics. In an exemplary embodiment, the value of the characterization algorithm calculated in substep 78 comprises the index used in the characterization step 72 for characterizing the sensor of interest mounted on the medical device.

In an exemplary embodiment, the processing step 70 further comprises a substep 80 of calculating, by the ECU, a respective scale for the spacing between a second sensor of interest and each sensor of a second subset of the sensors mounted on the medical device based on the respective magnitudes of the first and second configurations for the distances between the second sensor of interest and each respective sensor of the second subset of sensors. In such an embodiment, the processing step 70 further comprises a substep 82 of calculating, by the ECU, values for a plurality of metrics, at least one of which is based on one or more of the calculated scales calculated in substep 80. In such an embodiment, the processing step 70 may still further comprise a substep 84 of calculating, by the ECU, a value for a characterization algorithm for the second sensor of interest based on the values of the plurality of metrics calculated in substep 82. Finally, in an exemplary embodiment, the processing step 70 yet still further comprises a substep 86 of calculating, by the ECU, a value for a characterization algorithm for the medical device based on the values of the characterization algorithms calculated for the sensors of interest in substeps 78 and 84. In an exemplary embodiment, the value of the characterization algorithm for the medical device calculated in substep 86 comprises the index used in characterization step 72 to characterize the medical device.

In another exemplary embodiment, rather than the first and second configurations each comprising magnitudes of the distances between the plurality of sensors, they comprise magnitudes of an angle corresponding to a mechanical bend of the medical device at a sensor of interest. In such an embodiment, the substep 76 of the processing step 70 comprises calculating, by the ECU, a value of a metric based on the respective magnitudes of the first and second configurations for the angle. In an exemplary embodiment, the value of the metric comprises the index used in the characterization step 72 for characterizing the sensor of interest.

It should be understood that the system 10, and particularly the ECU 34, as described above may include conventional processing apparatus known in the art, capable of executing pre-programmed instructions stored in an associated memory, all performing in accordance with the functionality described herein. It is contemplated that the methods described herein, including without limitation the method steps of embodiments of the invention, will be programmed in a preferred embodiment, with the resulting software being stored in an associated memory and where so described, may also constitute the means for performing such methods. Implementation of the invention, in software, in view of the foregoing enabling description, would require no more than routine application of programming skills by one of ordinary skill in the art. Such a system may further be of the type having both ROM, RAM, a combination of non-volatile and volatile (modifiable) memory so that the software can be stored and yet allow storage and processing of dynamically produced data and/or signals.

Although only certain embodiments have been described above with a certain degree of particularity, those skilled in the art could make numerous alterations to the disclosed embodiments without departing from the scope of this disclosure. Joinder references (e.g., attached, coupled, connected, and the like) are to be construed broadly and may include intermediate members between a connection of elements and relative movement between elements. As such, joinder references do not necessarily infer that two elements are directly connected/coupled and in fixed relation to each other. Additionally, terms such as electrically connected, electrically couple, and in communication are meant to be construed broadly to encompass both wired and wireless connections and communications. It is intended that all matter contained in the above description or shown in the accompanying drawings shall be interpreted as illustrative only and not limiting. Changes in detail or structure may be made without departing from the invention as defined in the appended claims.

What is claimed is:

1. A system for characterizing at least one of a medical device and one or more of a plurality of sensors mounted thereon, said apparatus comprising:
an electronic control unit (ECU) configured to:
acquire a first configuration for said medical device;
acquire a second configuration for said medical device, wherein said second configuration comprises a calculated configuration; and
process said first and second configurations together to calculate an index upon which a characterization of at least one of said medical device and at least one of said sensors mounted thereon can be based.

2. The system of claim 1, wherein said first configuration comprises a user-defined configuration.

3. The system of claim 1, wherein said first configuration comprises a calculated configuration.

4. The system of claim 1, wherein said second configuration is calculated based on position coordinates corresponding to respective locations of said plurality of sensors.

5. The system of claim 1, wherein said ECU is further configured to characterize said at least one of said medical device and said at least one of said sensors mounted thereon based on said index.

6. The system of claim 1, wherein said first and second configurations each comprise magnitudes of the distances between said plurality of sensors, and further wherein said index can be used to characterize one of said plurality of sensors, said ECU configured to process said first and second configurations by:
calculating a respective scale for the spacing between said one sensor and each sensor of a subset of said sensors based on the respective magnitudes of said first and second configurations for the distances between said one sensor and each sensor of said subset of sensors.

7. The system of claim 6, wherein said ECU is further configured to process said first and second configurations by:
calculating a value of a metric based on at least one of said calculated scales, wherein said value of said metric comprises said index.

8. The system of claim 6, wherein said ECU is further configured to process said first and second configurations by:
calculating values for a plurality of metrics, at least one of which is based on at least one of said calculated scales; and
calculating a value for a characterization algorithm based on said values of said plurality of metrics, said value of said characterization algorithm comprising said index.

9. The system of claim 1, wherein said first and second configurations each comprise magnitudes of the distances between said plurality of sensors, and further wherein said index can be used to characterize said medical device, said ECU configured to process said first and second configurations by:
calculating respective scales for the spacing between a first of said plurality of sensors and each sensor of a first subset of said sensors based on the respective magnitudes of said first and second configurations for the distances between said first sensor and each sensor of said first subset of sensors;
calculating values for a plurality of metrics for said first sensor, wherein at least one of said plurality of metrics is based on at least one of said calculated scales corresponding to said first sensor;
calculating a value for a characterization algorithm for said first sensor based on said values of said plurality of metrics corresponding to said first sensor;
calculating respective scales for the spacing between a second of said plurality of sensors and each sensor of a second subset of said sensors based on the respective magnitudes of said first and second configurations for the distances between said second sensor and each sensor of said second subset of sensors;
calculating values for a plurality of metrics for said second sensor, wherein at least one of said plurality of metrics is based on at least one of said calculated scales corresponding to said second sensor;
calculating a value for a characterization algorithm for said second sensor based on said values of said plurality of metrics corresponding to said second sensor; and
calculate a characterization algorithm for said medical device based on said values of said characterization algorithms of said first and second sensors, said value of said characterization algorithm for said medical device comprising said index.

10. The system of claim 1, wherein said index can be used to characterize one of said plurality of sensors, and further wherein said first and second configurations each comprise a magnitude of an angle corresponding to a mechanical bend of said medical device at said one sensor, said ECU configured to process said first and second configurations by:
calculating a value of a metric based on the respective magnitude of said first and second configurations for said angle, wherein said value of said metric comprises said index.

11. An article of manufacture, comprising:
a computer-readable storage medium having a computer program encoded thereon for characterizing at least one of a medical device and one or more of a plurality of sensors mounted thereon, said computer program including code for:
acquiring a first configuration for said medical device;
acquiring a second configuration for said medical device, wherein said second configuration comprises a calculated configuration; and
processing said first and second configurations together to calculate an index upon which a characterization of at least one of said medical device and at least one of said sensors mounted thereon can be based.

12. The article of manufacture of claim 11, wherein said computer program further includes code for characterizing said at least one of said medical device and said at least one of said sensors mounted thereon based on said index.

13. The article of manufacture of claim 11, wherein said index can be used to characterize one of said plurality of sensors, and further wherein said first and second configurations each comprise magnitudes of the distances between said plurality of sensors, said code for processing said first and second configurations including code for:
calculating respective scales for the spacing between said one sensor and each sensor of a subset of said plurality of sensors based on the respective magnitudes of said first and second configurations for the distances between said one sensor and each sensor of said subset of sensors.

14. The article of manufacture of claim 13, wherein said code for processing said first and second configurations together further includes code for:
calculating a value of a metric based on at least one of said calculated scale, wherein said value of said metric comprises said index.

15. The article of manufacture of claim 13, wherein said code for processing said first and second configurations together further includes code for:
calculating values for a plurality of metrics, at least one of which is based on at least one of said calculated scales; and
calculating a value for a characterization algorithm based on said values of said metrics, wherein said value of said characterization algorithm comprises said index.

16. The article of manufacture of claim 11, wherein said index can be used to characterize said medical device, and further wherein said first and second configurations each comprise magnitudes of the distances between said plurality of sensors, said code for processing said first and second configurations together including code for:
calculating respective scales for the spacing between said a first sensor of said plurality of sensors and each sensor of a first subset of said sensors based on the respective magnitudes of said first and second configurations for the distances between said first sensor and each sensor of said first subset of sensors;
calculating values for a plurality of metrics for said first sensor, wherein at least one of said plurality of metrics is based on at least one of said calculated scales corresponding to said first sensor;
calculating a value for a characterization algorithm for said first sensor based on said values of said plurality of metrics corresponding to said first sensor;
calculating respective scales for the spacing between a second sensor of said plurality of sensors and each sensor of a second subset of said sensors based on the respective magnitudes of said first and second configurations for the distances between said second sensor and each sensor of said second subset of sensors;
calculating values for a plurality of metrics for said second sensor, wherein at least one of said plurality of metrics is based on at least one of said calculated scales corresponding to said second sensor;
calculating a value for a characterization algorithm for said second sensor based on said values of said plurality of metrics corresponding to said second sensor; and
calculating a value for a characterization algorithm for said medical device based on said values of said characterization algorithms of said first and second sensors, said value of said characterization algorithm for said medical device comprising said index.

17. The article of manufacture of claim 11, wherein said index can be used to characterize one of said plurality of sensors, and further wherein said first and second configurations each comprise a magnitude of an angle corresponding to a mechanical bend of said medical device at said one sensor, said code for processing said first and second configurations together including code for:
calculating a value of a metric based on the respective magnitude of said first and second configurations for said angle, wherein said value of said metric comprises said index.

18. A method for characterizing at least one of a medical device and one or more sensors mounted thereon, said method comprising the steps of:
providing an electronic control unit (ECU);
acquiring, by said ECU, a first configuration for said medical device;
acquiring, by said ECU, a second configuration for said medical device, wherein said second configuration comprises a calculated configuration; and
processing, by said ECU, said first and second configurations to calculate an index upon which a characterization of at least one of said medical device and at least one of said sensors mounted thereon can be based.

19. The method of claim 18 further comprising the step of characterizing, by said ECU, said at least one of said medical device and said at least one of said sensors mounted thereon based on said index.

20. The method of claim 18, wherein said index can be used to characterize one of said plurality of sensors, and further wherein said first and second configurations each comprise magnitudes of the distances between said plurality of sensors, said processing step comprising:
calculating respective scales for the spacing between said one sensor and each sensor of a subset of said plurality of sensors based on the respective magnitudes of said first and second configurations for the distances between said one sensor and each sensor of said subset of sensors.

21. The method of claim 20, wherein said processing step further comprises:
calculating a value of a metric based on at least one of said calculated scale, wherein said value of said metric comprises said index.

22. The article of manufacture of claim 20, wherein said processing step further comprises:
calculating values for a plurality of metrics, at least one of which is based on at least one of said calculated scales; and
calculating a value for a characterization algorithm based on said values of said metrics, wherein said value of said characterization algorithm comprises said index.

23. The article of manufacture of claim 18, wherein said index can be used to characterize said medical device, and further wherein said first and second configurations each comprise magnitudes of the distances between said plurality of sensors, and processing step comprising:
calculating respective scales for the spacing between said a first sensor of said plurality of sensors and each sensor of a first subset of said sensors based on the respective magnitudes of said first and second configurations for the distances between said first sensor and each sensor of said first subset of sensors;
calculating values for a plurality of metrics for said first sensor, wherein at least one of said plurality of metrics is based on at least one of said calculated scales corresponding to said first sensor;
calculating a value for a characterization algorithm for said first sensor based on said values of said plurality of metrics corresponding to said first sensor;
calculating respective scales for the spacing between a second sensor of said plurality of sensors and each sensor of a second subset of said sensors based on the respective magnitudes of said first and second configurations for the distances between said second sensor and each respective sensor of said second subset of sensors;
calculating values for a plurality of metrics for said second sensor, wherein at least one of said plurality of metrics is based on at least one of said calculated scales corresponding to said second sensor;
calculating a value for a characterization algorithm for said second sensor based on said values of said plurality of metrics corresponding to said second sensor; and
calculating a value for a characterization algorithm for said medical device based on said values of said characterization algorithms of said first and second sensors, said value of said characterization algorithm for said medical device comprising said index.

24. The article of manufacture of claim 18, wherein said index can be used to characterize one of said plurality of sensors, and further wherein said first and second configurations each comprise a magnitude of an angle corresponding to a mechanical bend of said medical device at said one sensor, said processing step comprises:
calculating a value of a metric based on the respective magnitude of said first and second configurations for said angle, wherein said value of said metric comprises said index.
